(19) United States
(12) Patent Application Publication
(10) Pub. No.: US 2024/0328990 A1
(43) Pub. Date: Oct. 3, 2024

(54) SYSTEMS AND METHODS FOR ANALYZING A TARGET MOLECULE

(71) Applicant: AXBIO INC., Santa Clara, CA (US)

(72) Inventors: Tao HONG, Santa Clara, CA (US); Igor Constantin IVANOV, Danville, CA (US); Hui TIAN, Cupertino, CA (US)

(21) Appl. No.: 18/732,126

(22) Filed: Jun. 3, 2024

Related U.S. Application Data
(63) Continuation of application No. PCT/US23/77338, filed on Oct. 19, 2023.
(60) Provisional application No. 63/418,204, filed on Oct. 21, 2022.

(51) Int. Cl. G01N 27/327; C12Q 1/6825; C12Q 1/6869; C12Q 1/6876

(52) U.S. Cl. CPC G01N 27/3278; C12Q 1/6825; C12Q 1/6869; C12Q 1/6876

(57) ABSTRACT

The present disclosure provides systems and methods for analyzing or identifying a target nucleic acid molecule. For example, a system of the present disclosure may comprise (a) a complex comprising a target nucleic acid molecule and a primer nucleic acid molecule, wherein the primer nucleic acid molecule comprises (i) a region that is complementary to a portion of the target nucleic acid molecule and (ii) an additional region that is non-complementary the target nucleic acid molecule, and (b) with said additional region having flown through a pore of a sensor, using said sensor to identify said additional region, thereby analyzing said target nucleic acid molecule. The sensor may be configured to detect one or more signals indicative of an impedance or impedance change in the sensor when the at least the portion of the target molecule is bound by the binding unit. The one or more signals may be usable to analyze or identify the target molecule.

### CROSS-REFERENCE

This application is a continuation of International Patent Application No. PCT/US23/77338, filed Oct. 19, 2023, which claims the benefit of U.S. Provisional Application No. 63/418,204, filed Oct. 21, 2022, each of which is entirely incorporated herein by reference.

### BACKGROUND

Nucleic acid sequencing may be used to provide sequence information for a nucleic acid sample. Such sequence information may be helpful in diagnosing or treating a subject (e.g., an individual, a patient, etc.) of a condition (e.g., a disease). For example, nucleic acid sequence information of a subject may be used to identify, diagnose, or develop a treatment for one or more genetic diseases. In another example, nucleic acid sequence information of one or more pathogens may lead to treatment for one or more contagious diseases.

Detection of one or more rare sequence variants (e.g., mutations) may be valuable for healthcare. Detection of rare sequence variants may be important for and early detection of one or more pathological mutations. Detection of one or more cancer-associated mutations (e.g., point mutations) in clinical samples may improve identification of one or more minimal residual diseases during chemotherapy or detection of tumor cells in relapsing patients. Additionally, such detection of the mutation(s) may be important for assessment of exposure to environmental mutagens, monitoring endogenous DNA repair, or studying accumulation of one or more somatic mutations in aging individuals. The detection or rare sequence variant(s) may enhance prenatal diagnosis and enable characterization of fetal cells present in maternal blood.

### SUMMARY

An aspect of the present disclosure provides a method for analyzing a target nucleic acid molecule, comprising: (a) providing a complex comprising the target nucleic acid molecule and a primer nucleic acid molecule, wherein the primer nucleic acid molecule comprises (i) a region that is complementary to a portion of the target nucleic acid molecule and (ii) an additional region that is non-complementary to the target nucleic acid molecule; and (b) with the additional region having flown through a pore of a sensor, using the sensor to identify the additional region, thereby analyzing the target nucleic acid molecule.

In some embodiments of any one of the methods provided herein, the method further comprises contacting the complex by an enzyme that is operatively coupled to the pore. In some embodiments of any one of the methods provided herein, the contacting effects flowing of the additional region through the pore.

In some embodiments of any one of the methods provided herein, the method further comprises in (b), alternating an electrical signal in the sensor to direct the additional region to flow through the pore two or more times. In some embodiments of any one of the methods provided herein, the method further comprises in (b): (i) subjecting the target nucleic acid molecule to an extension reaction from the primer nucleic molecule, to generate a growing strand that is coupled to the primer nucleic acid molecule, wherein the growing stand exhibits sequence complementarity to an additional portion of the target nucleic acid molecule; and (ii) obtaining sequence information of at least a portion of the growing stand by directing the at least the portion of the growing stand through the pore of the sensor.

In some embodiments of any one of the methods provided herein, in (a), the region of the primer nucleic acid molecule is flanked by (1) the additional region of the primer nucleic acid molecule and (2) the growing strand. In some embodiments of any one of the methods provided herein, in (b), the obtaining the sequence information comprises identifying a sequence read that comprises a distinctive sequence information that is indicative of at least a portion of the additional region of the primer nucleic acid molecule. In some embodiments of any one of the methods provided herein, in (b), the additional region of the primer nucleic acid molecule is directed through the pore prior to the at least the portion of the growing stand. In some embodiments of any one of the methods provided herein, in (b), the directing comprises alternating an electrical signal in the sensor to direct the at least the portion of the growing stand through the pore two or more times.

In some embodiments of any one of the methods provided herein, the additional region comprises at least 5 bases. In some embodiments of any one of the methods provided herein, of any one of the methods provided herein, the additional region comprises at least 10 bases. In some embodiments of any one of the methods provided herein, the additional region comprises at least 20 bases. In some embodiments of any one of the methods provided herein, the additional region comprises a net positive charge. In some embodiments of any one of the methods provided herein, the additional region comprises a net negative charge.

In some embodiments of any one of the methods provided herein, the pore is part of a nanopore protein. In some embodiments of any one of the methods provided herein, the pore is part of a solid-state nanopore. In some embodiments of any one of the methods provided herein, the target nucleic acid molecule is a circular nucleic acid molecule. In some embodiments of any one of the methods provided herein, in (c), the obtaining comprises detecting one or more signals indicative of an impedance or impedance change in the sensor when at least the additional portion is directed through the pore. In some embodiments of any one of the methods provided herein, the pore is embedded in a membrane. In some embodiments of any one of the methods provided herein, the primer nucleic acid molecule comprises a barcode.

Another aspect of the present disclosure provides a system for analyzing a target nucleic acid molecule, comprising: a primer nucleic acid molecule comprising (i) a region that is complementary to a portion of the target nucleic acid molecule and (ii) an additional region that is non-complementary to the target nucleic acid molecule, wherein the primer nucleic acid molecule is configured to form a complex with the target nucleic acid molecule; a sensor comprising a pore, wherein the sensor is configured to direct flow of the additional region through the pore; and a controller operatively coupled to the sensor, wherein the controller is configured to identify the addition region that is flown through the pore, thereby analyzing the target nucleic acid molecule.

In some embodiments of any one of the systems provided herein, the system further comprises an enzyme operatively coupled to the pore, wherein the enzyme is configured to contact the complex. In some embodiments of any one of the systems provided herein, the contact between the enzyme and the complex is configured to effect flowing of the additional region through the pore. In some embodiments of any one of the systems provided herein, the controller is further configured to alternate an electrical signal in the sensor to direct the additional region to flow through the pore two or more times.

In some embodiments of any one of the systems provided herein, the sensor is further configured to subject the target nucleic acid molecule to an extension reaction from the primer nucleic molecule, to generate a growing strand that is coupled to the primer nucleic acid molecule, wherein the growing stand exhibits sequence complementarity to an additional portion of the target nucleic acid molecule, and wherein the controller is further configured to obtain sequence information of at least a portion of the growing stand by directing the at least the portion of the growing stand through the pore of the sensor.

In some embodiments of any one of the systems provided herein, the region of the primer nucleic acid molecule is flanked by (1) the additional region of the primer nucleic acid molecule and (2) the growing strand. In some embodiments of any one of the systems provided herein, the controller is configured to identify a sequence read that comprises a distinctive sequence information that is indicative of at least a portion of the additional region of the primer nucleic acid molecule. In some embodiments of any one of the systems provided herein, the additional region of the primer nucleic acid molecule is directed through the pore prior to the at least the portion of the growing stand. In some embodiments of any one of the systems provided herein, the controller is configured to alternate an electrical signal in the sensor to direct the at least the portion of the growing stand through the pore two or more times.

In some embodiments of any one of the systems provided herein, the additional region comprises at least 5 bases. In some embodiments of any one of the systems provided herein, the additional region comprises at least 10 bases. In some embodiments of any one of the systems provided herein, the additional region comprises at least 20 bases. In some embodiments of any one of the systems provided herein, the additional region comprises a net positive charge. In some embodiments of any one of the systems provided herein, the additional region comprises a net negative charge.

In some embodiments of any one of the systems provided herein, the pore is part of a nanopore protein. In some embodiments of any one of the systems provided herein, the pore is part of a solid-state nanopore. In some embodiments of any one of the systems provided herein, the target nucleic acid molecule is a circular nucleic acid molecule. In some embodiments of any one of the systems provided herein, the controller is configured to detect one or more signals indicative of an impedance or impedance change in the sensor when at least the additional portion is directed through the pore. In some embodiments of any one of the systems provided herein, the pore is embedded in a membrane. In some embodiments of any one of the systems provided herein, the primer nucleic acid molecule comprises a barcode.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example of a sensor for analyzing or identifying a target molecule.

FIG. 2 shows a computer system that is programmed or otherwise configured to implement methods provided herein.

FIG. 3 shows an example process of analyzing or identifying a target molecule.

FIG. 4 shows an additional example process of analyzing or identifying a target molecule.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in the specification and claims, the singular forms “a,” “an,” and “the” can include plural references unless the context clearly dictates otherwise. For example, the term “a transmembrane receptor” can include a plurality of transmembrane receptors.

The terms “about” and “approximately,” as used interchangeably herein, generally refer to within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold of a value. Where particular values are described in the application and claims, unless otherwise stated, the term “about” can mean within an acceptable error range for the particular value.

The term “dielectric material,” as used herein, generally refers to an electrical insulating material that may be polarized by the action of an applied electric field. When a dielectric material is placed in electric field, electric charges may not flow through the dielectric material. In some cases, in such electric field, the dielectric material may exhibit dielectric polarization, in which positive charges may be displaced along the electric field and negative charges may shift in the opposite direction, thereby creating an internal electric field that may partly compensate the external electric field inside the dielectric material. Example of the dielectric material can include, but are not limited to, polyester, polyethylene, polypropylene, cloth (such as nylon), paper, laminate, glass, self-assembled monolayer (SAM), etc.

The term “biomolecule,” as used herein, generally refers to any molecule found in a biological system, a derivative thereof, or a functional variant thereof. The biomolecule may be naturally occurring or the result of an external disturbance of the system (e.g., a disease, poisoning, genetic manipulation, etc.), as well as synthetic analogs and derivatives thereof. Non-limiting examples of biomolecules may include amino acids (naturally occurring or synthetic), peptides, polypeptides, glycosylated and non-glycosylated proteins (e.g., polyclonal and monoclonal antibodies, receptors, interferons, enzymes, etc.), nucleosides, nucleotides, oligonucleotides (e.g., DNA, RNA, PNA oligos), polynucleotides (e.g., DNA, cDNA, RNA, etc.), carbohydrates, hormones, haptens, steroids, toxins, etc. Biomolecules may be isolated from natural sources, or they may be synthetic.

The term “cell,” as used herein, generally refers to a biological cell or cell derivative. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The terms “nucleotide,” “nucleobase,” and “base,” as used interchangeably herein, generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP), uridine triphosphate (UTP), and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein generally refers to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled (e.g., tagged with a label). Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink Deoxy Nucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

Naturally-occurring nucleotides guanine, cytosine, adenine, thymine, and uracil may be abbreviated as G, C, A, T, and U, respectively. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or a variant thereof) or a pyrimidine (i.e., C, T or U, or a variant thereof). A subunit can enable individual nucleic acid bases or groups of bases (e.g., AA, TA, AT, GC, CG, CT, TC, GT, TG, AC, CA, or uracil-counterparts thereof) to be resolved.

The terms “circularize” and “circularization,” as used herein, generally refer to the promotion or generation of a structure of a polynucleotide molecule with both of its ends coupled to each other (e.g., via covalent and/or hydrogen bonds). The ends of the polynucleotide molecule may be directly coupled to each other. Alternatively, the ends of the polynucleotide molecule may be indirectly coupled to each other via a coupling moiety (or a linker), e.g., at least one separate molecule capable of binding to each of the two ends of the polynucleotide molecule.

The terms “polynucleotide,” “oligonucleotide,” “oligomer,” and “nucleic acid,” as used interchangeably herein, generally refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure and can perform any function. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, complementary DNA (cDNA, such as double-strand cDNA (dd-cDNA) or single-stranded cDNA (ss-cDNA)), circulating tumor DNA (ctDNA), damaged DNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes (e.g., fluorescence in situ hybridization (FISH) probes), and primers. The sequence of nucleotides can be interrupted by non-nucleotide components. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide can be further modified after polymerization, such as by conjugation with a labeling component.

The term “gene,” as used herein, generally refers to a nucleic acid (e.g., DNA such as genomic DNA and cDNA) and its corresponding nucleotide sequence that is involved in encoding an RNA transcript. The term “gene” as used herein with reference to genomic DNA may include intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. The genes may not encode a polypeptide, for example, ribosomal RNA genes (rRNA) and transfer RNA (tRNA) genes. The term “gene” may not only include the transcribed sequences, but also non-transcribed regions including upstream and downstream regulatory regions, enhancers and promoters. A gene may be an “endogenous gene” or a native gene in its natural location in the genome of an organism. A gene may be an “exogenous gene” or a non-native gene. A non-native gene may be a gene not normally found in the host organism but which is introduced into the host organism by gene transfer (e.g., transgene). A non-native gene may be a naturally occurring nucleic acid or polypeptide sequence that comprises mutations, insertions and/or deletions (e.g., non-native sequence).

The term “mutation,” as used herein, generally refers to a change in the sequence of nucleotides of a normally conserved nucleic acid sequence resulting in the formation of a mutant as differentiated from the normal (unaltered) or wild type sequence. A position (e.g., relative to a gene or a sample polynucleotide) and sequence of the mutation may be undetermined prior to sequencing. Alternatively, a position (e.g., relative to a gene or a sample polynucleotide) and sequence of the mutation may be determined prior to sequencing, in which case the sequencing may be performed to detect a presence or absence of the mutation in the sample polynucleotide. A mutation can comprise a base-pair substitution (e.g. single nucleotide substitution) and a frame-shift mutation. The frame-shift mutation may require insertion or deletion of one to several nucleotide pairs.

The term “probe,” as used herein, generally refers to a nucleotide or polynucleotide that is tagged with a maker (e.g., a fluorescent marker) useful for detecting or identifying its corresponding target nucleotide or polynucleotide in a hybridization reaction by hybridization with a corresponding target sequence. The terms “nucleotide probe, “nucleotide tag,” and “tagged nucleotide,” as used interchangeable herein, generally refer to a probe having a single nucleotide. The terms “polynucleotide probe, “polynucleotide tag,” and “tagged polynucleotide,” as used interchangeable herein, generally refer to a probe having polynucleotide. A polynucleotide probe may be tagged with at least one marker (e.g., one marker per each nucleotide of the polynucleotide probe). A probe may be hybridizable to one or more target nucleotides or polynucleotides. A polynucleotide probe can be entirely complementary to one or more target polynucleotides in a sample or contain one or more nucleotides that are not complementary (i.e., a mismatch) to one or more nucleotides of the one or more target polynucleotides in the sample.

In some embodiments, the maker may be a redox species. The term “redox species,” as used herein, generally refers to a molecule or compound or a portion thereof (e.g., a molecular or functional moiety of a molecular or compound) that can be oxidized and/or reduced (i.e., “redox”) during or upon electrical stimulation (e.g., during or upon application of an electrical potential), or can undergo a Faradaic reaction. In an example, a redox species may comprise one or more molecular moieties that accept and/or donate one or more electrons depending on its redox state. In some cases, the redox species may form part (e.g., a molecular moiety) of a small molecule, a compound, a polymer molecule, or can exist as an individual molecule or compound. Examples of the redox species may include imidazolium, pyrrolidinium, tetraalkylammonium, [OTf]-, [FAP]-, [PF6]-, [BF4]-, [DCA]-, [NTf2]-, [FSI]-, [B(CN)4]-, ferrocene (Fc), derivatives thereof, functional variants thereof, and combinations thereof. Examples of Fc derivatives may include, methyl ferrocene, dimethyl ferrocene, ethyl ferrocene, propyl ferrocene, n-butyl ferrocene, t-butyl ferrocene, and 1,1-dicarboxylate ferrocene.

Identical nucleotides may be tagged with a same marker. Alternatively, identical nucleotides may be tagged with different markers. For example, a first nucleotide A may be tagged with a first maker, and a second nucleotide A may be tagged with a second maker, wherein the first maker and the second maker are different. In cases where a sensor can detect and distinguish the first maker and the second maker apart from each other, using a plurality of makers for the same nucleotide may help resolve sequencing of identical nucleotides that are presented in a sequential manner.

The terms “complement,” “complements,” “complementary,” and “complementarity,” as used interchangeably herein, generally refer to a sequence that is fully complementary to and hybridizable to the given sequence. A sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least and 100% sequence complementarity. The respective lengths may comprise a region of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides.

Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods

The term “hybridization” as used herein, generally refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence may be referred to as the “complement” of the first sequence. The term “hybridizable,” as applied to a polynucleotide, generally refers to the ability of the polynucleotide to form a complex that is stabilized via hydrogen bonding between the bases of the nucleotide residues in a hybridization reaction.

The term “target polynucleotide,” as used herein, generally refers to a nucleic acid molecule or polynucleotide in a population of nucleic acid molecules having a target sequence. in which the presence, amount, and/or nucleotide sequence, or changes in one or more of these, are desired to be determined. The term “target sequence” generally refers to a nucleic acid sequence on a single strand of nucleic acid. The target sequence may be a portion of a gene, a regulatory sequence, genomic DNA, cDNA, ctDNA, RNA including mRNA, miRNA, rRNA, or others. The target sequence may be a target sequence from a sample or a secondary target such as a product of an amplification reaction. The target polynucleotide may be part of a gene (or a fragment thereof) that comprises one or more mutations.

The term “stringent condition,” as used herein, generally refers to one or more hybridization conditions under which a nucleic acid having complementarity to a target sequence predominantly hybridizes with a target sequence, and substantially does not hybridize to non-target sequences. Stringent conditions may be sequence-dependent and may vary depending on a number of factors. In some cases, the longer the sequence, the higher the temperature at which the sequence may specifically hybridize to its target sequence.

The term “polymerase,” as used herein, generally refers to an enzyme (e.g., natural or synthetic) capable of catalyzing a polymerization reaction. Examples of polymerases can include a nucleic acid polymerase (e.g., a DNA polymerase or a RNA polymerase) and a transcriptase (e.g., a reverse transcriptase). A polymerase can be a polymerization enzyme. The term “DNA polymerase” generally refers to an enzyme capable of catalyzing a polymerization reaction of DNA.

The term “linked polymerase,” as used herein, generally refers to a polymerase such as a DNA polymerase that is coupled to (e.g., fused to) a linker. The linker may be capable of coupling to (e.g., binding or conjugating to) another entity (e.g., a nanopore, such as a protein nanopore or a solid state nanopore).

The terms “sequence variant” and “sequencing variant,” as used interchangeably herein, generally refer to any variation in sequence relative to one or more reference sequences. Typically, a sequence variant occurs with a lower frequency than a reference sequence for a given population of individuals for whom the reference sequence is provided. For example, a particular bacterial genus may have a consensus reference sequence for the 16S rRNA gene, but individual species within that genus may have one or more sequence variants within the gene or a portion of a gene that are useful in identifying that species in a population of bacteria. As a further example, sequences for multiple individuals of the same species or multiple sequencing reads for the same individual may produce a consensus sequence when optimally aligned, and sequence variants with respect to that consensus may be used to identify mutants in the population indicative of dangerous contamination. In general, a “consensus sequence” refers to a nucleotide sequence that reflects the most common choice of base at each position in the sequence where the series of related nucleic acids has been subjected to intensive mathematical and/or sequence analysis, such as optimal sequence alignment according to any of a variety of sequence alignment algorithms. A reference sequence may be a single reference sequence, such as a predetermined genomic sequence of a single individual. A reference sequence can be a consensus sequence formed by aligning multiple sequences, such as predetermined genomic sequences of multiple individuals serving as a reference population, or multiple sequencing reads of polynucleotides from the same individual. A reference sequence can be a consensus sequence formed by optimally aligning the sequences from a sample under analysis, such that a sequence variant represents a variation relative to corresponding sequences in the same sample. A sequence variant can occur with a low frequency in the population (also referred to as a “rare” sequence variant). For example, a sequence variant may occur with a frequency of or less than 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. A sequence variant can occur with a frequency of or less than 0.1%.

A sequence variant can be any variation with respect to a reference sequence. A sequence variation may consist of a change in, insertion of, or deletion of a single nucleotide, or of a plurality of nucleotides such as, for example, 2, 3, 4, 5, 6, 7, 8, 9, 10, or more nucleotides. Where a sequence variant comprises two or more nucleotide differences, the nucleotides that are different may be contiguous with one another or discontinuous. Examples of types of sequence variants include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g., methylation differences).

The term “sequencing,” as used herein, generally refers to a procedure for determining the order in which nucleotides occur in a target nucleotide sequence. Methods of sequencing can comprise high-throughput sequencing, such as, for example, next-generation sequencing (NGS). Sequencing may be whole-genome sequencing or targeted sequencing. Sequencing may be single molecule sequencing or massively parallel sequencing. Next-generation sequencing methods can be useful in obtaining millions of sequences in a single run. In an example, sequencing may be performed using one or more nanopore sequencing methods, e.g., sequencing-by-synthesis, sequencing-by-ligation, or sequencing-by-cleavage.

The term “nanopore,” as used herein, generally refers to a pore, channel, or passage formed or otherwise provided in a membrane. The membrane may be an organic membrane, such as a lipid bilayer, or a synthetic membrane, such as a membrane formed of a polymeric material such as a protein nanopore. The membrane may be a solid state membrane (e.g., silicon substrate). The nanopore may be disposed adjacent or in proximity to a sensing circuit or an electrode coupled to a sensing circuit, such as, for example, a complementary metal-oxide semiconductor (CMOS) or field effect transistor (FET) circuit. The nanopore may be part of the sensing circuit. A nanopore can have a characteristic width or diameter, for example, on the order of about 0.1 nanometer (nm) to 1000 nm. A nanopore can be a biological nanopore, solid state nanopore, hybrid biological-solid state nanopore, a variation thereof, or a combination thereof. Examples of the biological nanopore include, but are not limited to, OmpG from E. coli, sp., Salmonella sp., Shigella sp., and Pseudomonas sp., and alpha hemolysin (α-hemolysin) from S. aureus sp., MspA from M. smegmatis sp, a functional variant thereof, or a combination thereof. Sequencing may comprise forward sequencing and/or reverse sequencing. Examples of the solid state nanopore include, but are not limited to, silicon nitride, silicon oxide, graphene, molybdenum sulfide, a functional variant thereof, or a combination thereof. The solid state nanopore may be fabricated by high-energy beam manufacturing, imprinting (e.g., nanoimprinting), laser ablation, chemical etching, plasma etching (e.g., oxygen plasma etching), etc.

The term “nanopore sequencing complex,” as used herein, generally refers to a nanopore linked or coupled to an enzyme, e.g., a polymerase, which in turn is associated with a polymer, e.g., a polynucleotide template. The nanopore sequencing complex may be positioned in a membrane, e.g., a lipid bilayer, where it functions to identify polymer components, e.g., nucleotides or amino acids.

The terms “nanopore sequencing” and “nanopore-based sequencing,” as used interchangeably herein, generally refer to a method that determines the sequence of a polynucleotide with the aid of a nanopore. In some cases, the sequence of the polynucleotide may be determined in a template-dependent manner. In some cases, the methods, systems, or compositions disclosed herein may not be limited to any particular nanopore sequencing method, system, or device.

The term “barcode,” as used herein, generally refers to a predetermined nucleic acid sequence that allows some feature of a polynucleotide with which the barcode is associated to (e.g., a polynucleotide comprising at least a portion of the barcode or a polynucleotide having complementarity to at least a portion of the barcode) be identified. In some examples, the feature of the polynucleotide to be identified may be the sample from which the polynucleotide is derived. A barcode may be at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, or more nucleotides in length. A barcode may be at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, or at most about 2 nucleotides in length. A barcode associated with polynucleotides from a first sample may be different (e.g., different sequences and/or different lengths) than the barcode associated with polynucleotides from a second sample that is different than the first sample. In such a case, identification of the barcode in the respective polynucleotides may help identify the sample source of one or more of the polynucleotides. Thus, different samples with different barcodes can be analyzed (e.g., sequenced) together (e.g., in the batch), and separated during analysis based at least in part on the barcode. In some examples, a barcode may be identified accurately even after mutation, insertion, or deletion of one or more nucleotides in the barcode sequence (e.g., the mutation, insertion, or deletion of at least 1, at least 2, at least 3, at least 4, at least 5, at least 6, at least 7, at least 8, at least 9, at least 10, or more nucleotides). A plurality of polynucleotides from the same sample may have the same barcode. Alternatively, the plurality of polynucleotides from the same sample may have different barcodes. A first barcode may differ from a second barcode by at least three nucleotide positions, such as at least 3, at least 4, at least 5, at least 6, at least 7, at least 8, at least 9, at least 10, or more nucleotide positions. A plurality of barcodes may be represented in a pool of samples, each sample comprising polynucleotides comprising one or more barcodes that differ from the barcodes contained in the polynucleotides derived from the other samples in the pool. Samples of polynucleotides comprising one or more barcodes can be pooled based on the barcode sequences to which they are joined, such that all four of the nucleotide bases A, G, C, and T are approximately evenly represented at one or more positions along each barcode in the pool (such as at least 1, at least 2, at least 3, at least 4, at least 5, at least 6, at least 7, at least 8, at least 9, at least 10 or more positions, or all positions of the barcode). In some examples, the methods of the present disclosure may comprise identifying the sample from which a target polynucleotide is derived based on a barcode sequence to which the target polynucleotide is joined. The barcode may comprise a nucleic acid sequence that when joined to a target polynucleotide may serve as an identifier of the sample from which the target polynucleotide was derived. In an example, an oligonucleotide primer (e.g., an amplification primer) may comprise one or more barcodes. In another example, a nucleic acid molecule may be coupled (e.g., ligated) to an adaptor nucleic acid (e.g., for circularization), and the adaptor nucleic acid may comprise one or more barcodes. A barcode can be a non-natural polynucleotide sequence, e.g., can be a synthetic polynucleotide sequence.

The terms “real-time” and “real time,” as used interchangeably herein, generally refer to an event (e.g., an operation, a process, a measurement, a detection, etc.) that is performed almost immediately after or within a short period of time after another event (e.g., addition of a nucleobase, generation of a growing strand, etc.), such as within at least about 0.0001 millisecond (ms), at least about 0.0005 ms, at least about 0.001 ms, at least about 0.005 ms, at least about 0.01 ms, at least about 0.05 ms, at least about 0.1 ms, at least about 0.5 ms, at least about 1 ms, at least about 5 ms, at least about 0.01 seconds, at least about 0.05 seconds, at least about 0.1 seconds, at least about 0.5 seconds, at least about 1 second, or more. In some cases, a real time event may be performed almost immediately after or within a short period of time after another event, such as within at most about 1 second, at most about 0.5 seconds, at most about 0.1 seconds, at most about 0.05 seconds, at most about 0.01 seconds, at most about 5 ms, at most about 1 ms, at most about 0.5 ms, at most about 0.1 ms, at most about 0.05 ms, at most about 0.01 ms, at most about 0.005 ms, at most about 0.001 ms, at most about 0.0005 ms, at most about 0.0001 ms, or less.

The term “sample,” as used herein, generally refers to any sample that may include one or more constituents (e.g., nucleic acid molecules) for processing or analysis. The sample may be a biological sample. The sample may be a cellular or tissue sample. The sample may be a cell-free sample, such as blood (e.g., whole blood), plasma, serum, sweat, saliva, or urine. The sample may be obtained in vivo or cultured in vitro.

The term “subject,” as used herein, generally refers to an individual or entity from which a sample is derived, such as, for example, a vertebrate (e.g., a mammal, such as a human) or an invertebrate. A mammal may be a murine, simian, human, farm animal (e.g., cow, goat, pig, or chicken), or a pet (e.g., cat or dog). The subject may be a plant. The subject may be a patient. The subject may be asymptomatic with respect to a disease (e.g., cancer). Alternatively, the subject may be symptomatic with respect to the disease.

The term “deconvolution,” as used herein, generally refers to identification of an individual (e.g., a single nucleotide, or a specific sequence of nucleotides) in a pool or library by detection of the presence of a known associated indicator (e.g., property, label, identifier, etc.). In some cases, deconvolution of data measured by a sensor as provided herein may be utilized to identify (i) at least a portion of a non-complementary region of a primer nucleic acid molecule and/or (ii) one or more nucleotides in a growing strand that is at least partially complementary to a target nucleic acid molecule. A deconvolution function utilized for such deconvolution can be a fixed deconvolution function. Alternatively or in addition to, a deconvolution function can be derived as part of a best fit algorithm.

For example, a first sequence of nucleotides (e.g., ACCT) can generate a first sensor signal (e.g., impedance signal) having a first sensing signature, and another sequence of nucleotides (e.g., GACC) can generate a second sensor signal having a second sensing signature. When the sensor detects the first sensor signal, the deconvolution method can identify (e.g., with a degree of certainty) that it is the first sensor signal having the first sensing signature, and thus can deconvolute the first sensor signal and assign the first sequence of nucleotides (e.g., ACCT) as the detected base sequence. Similarly, when the sensor detects the second sensor signal, the deconvolution method can identify (e.g., with a degree of certainty) that it is the second sensor signal having the second sensing signature, and thus can deconvolute the second sensor signal and assign the second sequence of nucleotides (e.g., GACC) as the detected base sequence.

### I. Systems and Methods for Detection and Analysis of a Target Molecule

In an aspect, the present disclosure provides a method for processing (e.g., analyzing) a target nucleic acid (NA) molecule. The method can comprise providing a complex (e.g., a nucleic acid molecule complex) comprising the target nucleic acid molecule and a primer nucleic acid (NA) molecule. The primer nucleic acid molecule can comprise a plurality of regions. The plurality of regions can comprise (i) a region that is complementary to a portion of the target nucleic acid molecule and (ii) an additional region that is non-complementary to the target nucleic acid molecule. The method can comprise, with the additional region having flown through a pore of a sensor, using the sensor to identify the additional region, thereby analyzing the target nucleic acid molecule.

The target nucleic acid molecule can be from a nucleic acid sample (NA sample). The NA sample can be a biological sample (e.g., at least a portion of a bodily sample from a subject), and the target nucleic acid molecule can be at least a portion of the biological sample (e.g., without further ex vivo processing such as amplification via, for example, polymerase chain reaction). For example, the target nucleic acid molecule can be at least a portion of a natural nucleic acid molecule from the biological sample (e.g., a chromosomal fragment, a cell free DNA, etc.). Alternatively, the target nucleic acid molecule can be a synthetic molecule derived from the biological sample (e.g., a copied, amplified, or modified variant of a natural nucleic acid molecule). The target nucleic acid molecule can be a linear NA molecule or a circular NA molecule.

In the complex, the target NA molecule and the primer NA molecule can be bound together. The target NA molecule and the primer NA molecule may be heterologous to one another (e.g., not from a same source, or not from a same biological sample of a subject). Alternatively, the target NA molecule and the primer NA molecule may be homologous. The target NA molecule and the primer NA molecule may be bound by covalent bonds. Alternatively or in addition to, the target NA molecule and the primer NA molecule may be bound by ionic bonds. Alternatively, or in addition to, the target NA molecule and the primer NA molecule may be bound by hydrogen bonds.

The target NA molecule and the primer NA molecule may be fully complementary. Alternatively, the target NA molecule and the primer NA molecule may be partially complementary (e.g., there may be at least one region of the primer NA molecule that is complementary and at least one region of the primer NA molecule that is non-complementary to the target NA molecule, and/or there may be at least one region of the target NA molecule that is complementary and at least one region of the NA molecule that is non-complementary to the primer NA molecule) but not entirely complementary to one another.

A non-complementary region (e.g., that of the target NA molecule or the primer NA molecule, e.g., when compared to one another) can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 nucleobases (or bases, as used interchangeably herein) in length. The non-complementary region can comprise at most about 100, at most about 95, at most about 90, at most about 85, at most about 80, at most about 75, at most about 70, at most about 65, at most about 60, at most about 55, at most about 50, at most about 45, at most about 40, at most about 35, at most about 30, at most about 25, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 base in length. In some cases, the non-complementary region can comprise a plurality of bases as provided herein, and the plurality of bases can be a contiguous polynucleotide sequence or a non-contiguous polynucleotide sequence.

A complementary region (e.g., that of the target NA molecule or the primer NA molecule, e.g., when compared to one another) can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 nucleobases (or bases, as used interchangeably herein) in length. The complementary region can comprise at most about 100, at most about 95, at most about 90, at most about 85, at most about 80, at most about 75, at most about 70, at most about 65, at most about 60, at most about 55, at most about 50, at most about 45, at most about 40, at most about 35, at most about 30, at most about 25, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 base in length. In some cases, the complementary region can comprise a plurality of bases as provided herein, and the plurality of bases can be a contiguous polynucleotide sequence or a non-contiguous polynucleotide sequence.

The complementary region of the primer NA molecule may be downstream of the non-complementary region of the primer NA molecule (e.g., after the 3′ end of the non-complementary region of the primer NA molecule). Alternatively, the complementary region of the primer NA molecule may be upstream of the non-complementary region of the primer NA molecule (e.g., before the 5′ end of the non-complementary region of the primer NA molecule).

The complementary region of the primer NA molecule can be substantially (e.g., entirely) complementary to a portion of the target nucleic acid molecule, e.g., thereby forming a complementary, double stranded portion in the complex. Alternatively, the complementary region of the primer NA molecule can comprise a consecutive polynucleotide sequence that is not completely complementary (e.g., forming A to T or U pairs, and G to C pairs across the entire consecutive polynucleotide sequence) to a portion of the target nucleic acid molecule. For example, in the primer NA molecule, the consecutive polynucleotide sequence that is not complementary to the target NA molecule can be disposed between two NA regions (e.g., two consecutive polynucleotide sequences) that are each complementary to different portions of the target NA molecule. A length of such consecutive polynucleotide sequence of the primer NA molecule can be at least about 2, at least about 3, at least about 4, at least about 5, at least about 6, at least about 7, at least about 8, at least about 9, or at least about 10, at least about 12, at least about 13, at least about 14, at least about 15, at least about 20, at least about 25, at least about 30, at least about 35, at least about 40, at least about 45, at least about 50, at least about 60, at least about 80, at least about 100, or more nucleotides. The length of the consecutive polynucleotide sequence of the primer NA molecule can be at most about 100, at most about 80, at most about 60, at most about 50, at most about 45, at most about 40, at most about 35, at most about 30, at most about 25, at most about 20, at most about 15, at most about 14, at most about 13, at most about 12, at most about 11, at most about 10, at most about 9, at most about 8, at most about 7, at most about 6, at most about 5, at most about 4, at most about 3, or at most about 2 nucleotides. Alternatively or in addition to, the consecutive polynucleotide sequence of the primer NA molecule can be partially complementary, e.g., forming complementary pairs for less than a threshold portion (e.g., less than about 50%, less than about 40%, less than about 30%, less than about 20%, less than about 10%, less than about 5%, or less) of the consecutive polynucleotide sequence with a portion of the target nucleic acid molecule.

The primer NA molecule may have one or more regions (e.g., one or more polynucleotide regions that are not directly adjacent to one another) of non-complementarity to a target NA molecule. The primer may have at least about 1, at least about 2, at least about 3, at least about 4, at least about 5, at least about 6, at least about 7, at least about 8, at least about 9, or at least about 10 regions of non-complementarity to the target NA molecule. The primer NA molecule may have at most about 10, at most about 9, at most about 8, at most about 7, at most about 6, at most about 5, at most about 4, at most about 3, at most about 2, or at most about 1 regions of non-complementarity to the target NA molecule.

The primer NA molecule as disclosed herein may comprise (e.g., as the additional region of the primer NA molecule, or as a portion of the additional region) n repeats of a single nucleotide (e.g., Aₙ, Tₙ, Cₙ, Gₙ, etc.). For example, the additional region of the primer NA molecule can comprise Tₙ, such as T₁₀, T₂₀, T₃₀, T₄₀, etc. Alternatively, or in addition to, the primer NA molecule may comprise n repeats of a polynucleotide sequence, wherein the polynucleotide sequence can be a dinucleotide (e.g., two different and contiguous nucleotides, such as (AT) n, (GC)ₙ, etc.), a trinucleotide, a tetranucleotide, a pentanucleotide, etc. In some examples, the primer NA molecule may comprise n repeats wherein n may be at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more. In some examples, the primer NA molecule may comprise n repeats wherein n may be at most 100, at most 90, at most 80, at most 70, at most 60, at most 50, at most 45, at most 40, at most 35, at most 30, at most 25, at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, or at most 2.

The primer NA molecule as disclosed herein may comprise (e.g., as the additional region of the primer NA molecule, or as a portion of the additional region) at least a portion of a barcode. The complementary region of the primer NA molecule may comprise the barcode or a portion of the barcode. Alternatively or in addition to, the non-complementary region of the primer NA molecule may comprise the barcode or a portion of the barcode. Primer NA molecules used for sequencing different target NA molecules may have the same barcode. Alternatively or in addition to, primer NA molecule used for sequencing different target NA molecules may have different barcodes. For example, a plurality of different barcode can be a set of unique polynucleotide sequences, such that (i) one primer NA molecule or a complementary product thereof can be distinguishable (e.g., upon analyzing sequencing results) from (ii) another primer NA molecule or a complementary product thereof, via their unique barcodes. The primer NA molecule may comprise at least 1, at least 2, at least 3, at least 4, at least 5, at least 6, at least 7, at least 8, at least 9, at least 10 or more barcodes. The primer NA molecule may comprise at most 10, at most 9, at most 8, at most 7, at most 6, at most 5, at most 4, at most 3, at most 2, or at most 1 barcode. Alternatively, the primer NA molecule may not comprise a barcode.

The primer NA molecule can facilitate an extension reaction of the target NA molecule (e.g., in presence of an enzyme, such as a polymerase), thereby generating a growing strand that is coupled to the primer NA molecule. The growing strand may be generated while the complex is disposed adjacent to the sensor (e.g., coupled to a sensor, such as being bound by a binding moiety-nanopore unit). Generation of the growing strand may be initiated prior to, during, or subsequent to flowing of at least a portion of the additional region through the pore of the sensor. The growing strand coupled to the primer NA molecule may be complementary to at least a portion of the target NA molecule. The growing strand coupled to the primer NA molecule may be partially complementary to the target NA molecule (e.g., has a portion that is non-complementary). In some examples, the complementary region of the primer NA molecule may be flanked by (e.g., disposed between) the non-complementary region of the primer NA molecule and the growing strand.

In the primer NA molecule (e.g., prior to forming the complex with the target nucleic acid molecule, prior to generation of the growing strand, etc.), a number of nucleobases in the region that is complementary to the portion of the target nucleic acid molecule can be greater than a number of nucleobases in the additional region that is non-complementary to the target nucleic acid molecule by at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 25, at least or up to about 30, at least or up to about 40, at least or up to about 50, at least or up to about 60, at least or up to about 70, at least or up to about 80, at least or up to about 90, or at least or up to about 100 nucleobase. In such primer NA molecule, a size of the region that is complementary to the portion of the target nucleic acid molecule can be greater than a size of the additional region that is non-complementary to the target nucleic acid molecule by at least or up to about 1%, at least or up to about 2%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 120%, at least or up to about 150%, at least or up to about 200%, at least or up to about 300%, at least or up to about 400%, at least or up to about 500%, or at least or up to about 1,000%.

Alternatively, in the primer NA molecule (e.g., prior to forming the complex with the target nucleic acid molecule, prior to generation of the growing strand, etc.), a number of nucleobases in the additional region that is non-complementary to the target nucleic acid molecule can be greater than a number of nucleobases in the region that is complementary to the portion of the target nucleic acid molecule by at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 25, at least or up to about 30, at least or up to about 40, at least or up to about 50, at least or up to about 60, at least or up to about 70, at least or up to about 80, at least or up to about 90, or at least or up to about 100 nucleobase. In such primer NA molecule, a size of the additional region that is non-complementary to the target nucleic acid molecule can be greater than a size of the region that is complementary to the portion of the target nucleic acid molecule by at least or up to about 1%, at least or up to about 2%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 120%, at least or up to about 150%, at least or up to about 200%, at least or up to about 300%, at least or up to about 400%, at least or up to about 500%, or at least or up to about 1,000%.

A nucleic acid molecule (e.g., the target NA molecule or the primer NA molecule) may be circular. Alternatively, a nucleic acid molecule may be non-circular. For example, a nucleic acid molecule may be linear. In some examples, one or both of the target NA molecule and the primer NA molecule in the complex may be linear. Alternatively or in addition to, one or both of the target NA molecule and the primer NA molecule in the complex may be circular. A nucleic acid molecule can be single-stranded. Alternatively, a nucleic acid molecule can be double-stranded.

A nucleic acid molecule (e.g., the target NA molecule or the primer NA molecule) may have a net positive charge. In some cases, the nucleic acid molecule may have a net charge of +1, +2, +3, +4, +5, +6, +7, +8, +9, or +10. Alternatively, the nucleic acid molecule may have a net negative charge. In some cases, the nucleic acid molecule may have a net charge of −1, −2, −3, −4, −5, −6, −7, −8, −9, or −10. Alternatively, the nucleic acid molecule may have a neutral charge (e.g., a charge of 0 or no charge). For example, the primer NA molecule of the NA molecule complex may have a net positive charge. Alternatively, the primer NA molecule of the NA molecule complex may have a net negative charge. Alternatively, the primer NA molecule of the NA molecule complex may have a neutral charge. In another example, the complementary portion of the of the NA molecule complex may have a net positive charge. Alternatively, the complementary portion of the of the NA molecule complex may have a net negative charge. Alternatively, the complementary portion of the of the NA molecule complex may have a neutral charge. In a different example, the non-complementary portion of the of the NA molecule complex may have a net positive charge. Alternatively, the non-complementary portion of the of the NA molecule complex may have a net negative charge. Alternatively, the non-complementary portion of the of the NA molecule complex may have a neutral charge.

The target nucleic acid molecule can be a circular NA molecule. The circular NA molecule can be derived from a linear NA molecule (e.g., a natural linear NA molecule, or a copied or modified variant thereof) that is circularized via an adapter (e.g., a synthetic polynucleotide sequence). Such circularization can be a ligation between the linear NA molecule and the adapter, e.g., a non-enzymatic, chemical conjugation or an enzymatic conjugation. At least a portion of the primer NA molecule can exhibit complementarity to at least a portion of the adapter. Alternatively, the primer NA molecule may not exhibit complementarity to the adapter. The adapter can comprise at least about 2, at least about 5, at least about 10, at least about 15, at least about 20, at least about 25, at least about 30, at least about 40, at least about 50, at least about 100, or more nucleobases. The adapter can comprise at most about 100, at most about 50, at most about 40, at most about 30, at most about 25, at most about 20, at most about 15, at most about 10, at most about 5, or at most about 2 nucleobases.

The method as provided herein can comprise processing (e.g., analyzing or identifying) a target molecule such as the target nucleic acid molecule. One or more sensors can be utilized for the processing. The sensor can be configured to detect one or more signals (e.g., current, voltage, impedance, or a change thereof in the sensor) when at least a portion of the target molecule is bound by or in proximity to at least a portion of the sensor. The one or more signals may be usable to analyze or identify the target molecule. For example, the one or more signals can be indicative of an impedance or impedance change in the sensor.

The one or more sensors can comprise at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1,000, or more sensors (e.g., within a single sensor chip, such as a single sequencing chip). the one or more sensors can comprise at most about 1,000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 10, at most about 9, at most about 8, at most about 7, at most about 6, at most about 5, at most about 4, at most about 3, at most about 2, or fewer sensor(s).

A detected signal from the one or more sensors (e.g., indicative of an impedance or impedance change in the sensor) that is induced by the target molecule maybe a single measurement. Alternatively, the detected signal may be a median or average of a plurality of measurements, such as at least about 2, at least about 3, at least about 4, at least about 5, at least about 6, at least about 7, at least about 8, at least about 9, at least about 10, at least about 15, at least about 20, or more measurements, or at most about 20, at most about 15, at most about 10, at most about 9, at most about 8, at most about 7, at most about 6, at most about 5, at most about 4, at most about 3, or at most about 2 measurements.

In some cases, the sensor may comprise a nanopore protein. The nanopore protein may comprise a pore. Non-limiting examples of nanopore proteins can include porins (e.g., MspA porins), hemolysins, aerolysins, transporter proteins, channel proteins, and proteins that comprise a B-barrel domain structure. In some cases, the sensor may comprise a solid-state nanopore (e.g., a non-proteinaceous nanopore).

In some cases, at least a portion of the nanopore may be embedded in a membrane. The membrane may be a lipid bilayer. Alternatively, the membrane may be a single lipid layer. In some examples, the membrane is a biological membrane (e.g., a cell membrane).

For (e.g., when) detecting the one or more signals (e.g., indicative of the impedance or impedance change in the sensor), at least a portion of the complex can be bound (e.g., directly or indirectly) by a binding moiety of the sensor. The binding moiety may be configured to bind the at least the portion of the target molecule (e.g., a nucleotide, an amino acid, a small molecule, an ion, etc.). Alternatively, or in addition to, the binding moiety may be configured to bind to at least a portion of the primer nucleic acid molecule. In an example, at least a portion of the complementary regions of the target nucleic acid molecule and the primer nucleic acid molecule can be bound by the binding moiety.

One sensor and one binding unit can be operatively coupled to one another as a single unit (e.g., a nanopore sensor-polymerase binding unit fusion polypeptide). In such cases, the number of the sensor(s) and the number of the binding unit(s) may be the same (e.g., within a single sensor chip, such as a single sequencing chip). Alternatively, a single sensor (e.g., a nanopore) can be operatively coupled to a plurality of binding units (e.g., a plurality of polymerases).

The binding moiety can comprise an enzyme, such that at least a portion of the nucleic acid complex may be contacted by the enzyme, e.g., prior to the additional region of the primer nucleic acid molecule flowing through the pore of the sensor. Non-limiting examples of such an enzyme can include a DNA polymerase, a RNA polymerase, a DNA primase, a DNA helicase, a DNA ligase, or a topoisomerase. The enzyme may be operatively coupled to the sensor (e.g., the nanopore of the sensor). The enzyme can be covalently coupled to the sensor (e.g., covalently linked to the sensor as a fusion polypeptide construct). Alternatively, the enzyme may be non-covalently coupled to the sensor.

The complex as provided herein can be formed prior to being contacted by the sensor (e.g., the binding moiety of the sensor). Alternatively, a portion (e.g., one of but not both of the target nucleic acid molecule and the primer nucleic acid molecule) of what would constitute the complex can be contacted by the sensor (e.g., the binding moiety of the sensor) prior to forming the complex.

Contacting of at least a portion of the NA complex by the enzyme may affect the flowing of a portion of the complex into the pore of the sensor. The portion of the complex that enters the pore of the sensor may be at least a portion of the target NA molecule. Alternatively, the portion of the complex that enters the pore of the sensor may be at least a portion of the primer NA molecule. In some cases, the portion of the primer NA molecule that enters the pore of the sensor may be the complementary region. Alternatively, the portion of the primer NA molecule that enters the pore of the sensor may be the non-complementary region. For example, upon binding of the at least the portion of the complex to the enzyme that is coupled to the nanopore, the non-complementary region of the primer nucleic acid molecule can be directed to enter the pore of the nanopore or to flow through the pore of the nanopore.

The NA molecule complex may be formed prior to the flowing of a portion of the complex into the pore of the sensor. Alternatively, the NA molecule complex may be formed after a portion of the complementary region of the nucleic acid flows into the pore.

In some cases, contacting of the NA complex by the binding moiety (e.g., the enzyme) may be sufficient to effect the flowing of a portion of the complex (e.g., the additional region of the primer NA molecule) into the pore of the sensor. For example, upon the contacting, the portion of the complex (e.g., a portion of the primer NA molecule, such as the additional region that is non-complementary) may naturally flow into the pore of the sensor, e.g., without any additional external force. Without wishing to be bound by theory, the additional, non-complementary region of the primer NA molecule may be attracted towards the pore of the sensor due at least in part to the close proximity between the additional, non-complementary region of the primer NA molecule and the pore of the sensor. Alternatively, the contacting may not be sufficient to effect the flowing, and an additional external force (e.g., electrical field between two electrodes that are disposed on opposite ends of the pore as provided herein) may be needed to drive the flowing.

In some cases, a portion of the complex that enters the pore (e.g., the non-complementary region of the primer nucleic acid molecule) can be recruited towards to pore or can be directed to enter the pore of the sensor via controlling the electrical properties of the sensor (e.g., controlling the electric field across the sensor). The electric field can be increased, thereby recruiting/attracting the portion of the complex towards and/or into the pore. Alternatively, the electric field can be decreased, thereby recruiting/attracting the portion of the complex towards and/or into the pore. In some cases, when the electric field is changed to recruit/attract the non-complementary region of the primer nucleic acid molecule towards the pore of the sensor, the electric field across the sensor may be continuously controlled (e.g., continuously increased or decreased) to effect at least a portion of (i) the remainder of the primer NA molecule and/or (ii) a growing strand coupled to the primer NA molecule to flow through the pore. Alternatively, the electric field may be kept constant while the at least the portion of (i) the remainder of the primer NA molecule and/or (ii) the growing strand to flow through the pore. Yet in another alternative, the electric field may not need to be controlled to permit the at least the portion of (i) the remainder of the primer NA molecule and/or (ii) the growing strand to flow through the pore (e.g., due to innate action of the pore such as the nanopore protein).

An electrical signal can be applied across the sensor as provided herein. The electrical signal of the sensor can be constant. Alternatively, the electrical signal across the sensor may be an alternating electrical signal. In some examples, the electrical signal or a change thereof may direct a portion of the NA complex to enter into the pore of the sensor. Alternatively, or in addition to, the electrical signal or a change thereof may direct a portion of the NA complex to flow through the pore of the sensor. Alternatively, or in addition to, the electrical signal or a change thereof may direct a portion of the NA complex to reverse its movement through the sensor (e.g., going towards a direction, and subsequently going towards an opposite direction). Alternatively, or in addition to, the electrical signal or a change thereof may direct a portion of the NA complex to stop movement relative to the pore of the sensor (e.g., become lodged or unmoving within the pore of the sensor).

The electrical signal or a change thereof may direct at least a portion of the primer NA molecule (e.g., the additional, non-complementary region) through the pore of the sensor for at least about 1, at least about 2, at least about 3, at least about 4, at least about 5, at least about 6, at least about 7, at least about 8, at least about 9, at least about 10, or more time(s). The electrical signal may direct at least a portion of the primer NA molecule through the pore of the sensor for at most about 10, at most about 9, at most about 8, at most about 7, at most about 6, at most about 5, at most about 4, at most about 3, at most about 2, or fewer time(s).

In an aspect, the present disclosure provides a system to implement any one of the methods provided herein. The system (e.g., a device) can comprise the sensor as provided herein, for analyzing the target nucleic acid molecule. The system can further comprise a controller for controlling one or more (or any) operations of analyzing the target nucleic acid molecule. The controller can comprise a computer processor. The controller may be operatively coupled to the sensor. The controller can be configured to The controller can be configured to control the electrical signal or change thereof across the sensor (e.g., across the nanopore). The controller can obtain data indicative of the one or more signal reading (e.g., current, voltage, impedance, or a change thereof in the sensor) from the sensor and identify (e.g., sequence) the nucleic acid molecule that flows through the pore based at least in part from analyzing the data. In some examples, the controller may identify the primer NA molecule (e.g., identify a barcode of the primer NA molecule). The controller may identify the portion of the primer which is non-complementary to the target NA molecule. Alternatively or in addition to, the controller may identify the portion of the primer NA molecule which is complementary to the target NA molecule. Alternatively or in addition to, the controller may identify the growing strand coupled to the primer molecule.

Alternatively or in addition to, the controller may not be operatively coupled to the sensor; instead, the controller may be operatively coupled to the membrane in which the sensor is embedded.

The controller may be configured to control the electrical signal of the sensor. The controller may be configured to turn on and turn off the electrical signal. The controller may be configured to change (e.g., increase or decrease) the electrical signal. The controller may be configured to alternate the electrical signal. The controller may be configured to maintain the electrical signal. The controller may be configured to identify a sequence read of the nucleic acid molecule flowing through the pore based at least in part on data indicative of the one or more signal reading from the sensor. The controller may identify a sequence read based on distinctive sequence information, e.g., based at least in part on data indicative of the one or more signal reading from the sensor.

The growing strand that is coupled to the primer NA molecule may be directed through the pore of the sensor. The non-complementary region of the primer NA molecule may be directed through the pore of the sensor prior to at least a portion of the growing strand entering and/or flowing through the pore of the sensor. Alternatively or in addition to, the non-complementary region of the primer NA molecule may be directed through the pore of the sensor after at least a portion of the growing strand enters and/or flows through the pore of the sensor. The non-complementary region of the primer NA molecule may be directed through the pore of the sensor prior to the complementary region of the primer NA molecule entering and/or flowing through the pore of the sensor. Alternatively or in addition to, the non-complementary region of the primer NA molecule may be directed through the pore of the sensor after the complementary region of the primer NA molecule enters or flows through the pore of the sensor. The complementary region of the primer NA molecule may be directed through the pore of the sensor prior to at least a portion of the growing strand entering and/or flowing through the pore of the sensor. Alternatively or in addition to, the complementary region of the primer NA molecule may be directed through the pore of the sensor after at least a portion of the growing strand enters or flows through the pore of the sensor.

For example, the non-complimentary region of the primer NA molecule, the complementary region of the primer NA molecule, and at least a portion of the growing strand coupled to the complementary region of the primer NA molecule (e.g., via the polymerase as provided herein) can enter and flow through the nanopore in a sequential manner (e.g., in the order as described herein), and accordingly, the controller may obtain the signal data from the nanopore sensor to identify (e.g., sequence) the non-complimentary region of the primer NA molecule, the complementary region of the primer NA molecule, and the at least the portion of the growing strand, respectively in a sequential manner.

Sequence information related to the target nucleic acid molecule may be obtained from the NA molecule complex. In some examples, the sequence information may be obtained through sequencing of at least a portion of the primer NA molecule (e.g., at least a portion of the non-complementary region) and at least a portion of the growing strand coupled to the primer NA molecule. In some examples, sequence information may be obtained of the entire primer NA molecule and the at least the portion of the growing strand.

Sequence information may comprise identification of a sequence read from the data indicative of the one or more signal reading from the sensor. A sequence read may have distinctive sequence information that is indicative of at least a portion of the non-complementary region of the primer NA molecule. Alternatively or in addition to, a sequence read may have distinctive sequence information that is indicative of at least a portion of the complementary region of the primer NA molecule and/or the growing strand coupled to the primer NA molecule.

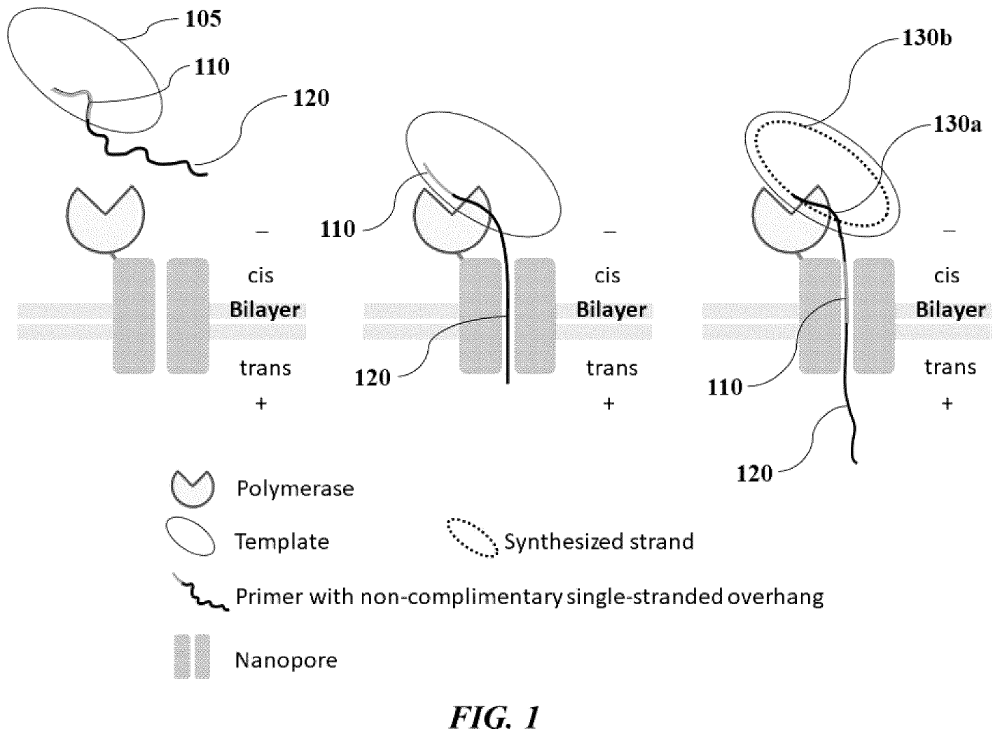

FIG. 1 schematically illustrates an example of the system (e.g., for direct-read sequencing) comprising a nanopore embedded in a membrane bilayer, and methods thereof. Operatively coupled to the nanopore is a polymerase. A target nucleic acid molecule (e.g., a circular template nucleic acid molecule) 105 is coupled to (e.g., via annealing) a linear primer nucleic acid molecule, which comprises (i) a complementary region 110 with respect to at least a portion of the target nucleic acid molecule and (ii) a non-complementary single strand overhang 120, to form a nucleic acid complex. See left panel in FIG. 1. The nucleic acid complex is contacted by the polymerase that is coupled to a nanopore sensor. Prior to, during, or subsequent to the contact between the nucleic acid complex and the polymerase, the non-complementary region 120 enters through the nanopore, and the complementary region 110 enters through the nanopore subsequent to the entry of the non-complementary region 120. See middle panel in FIG. 1. Based on the template, the polymerase synthesizes a growing strand (130a and 130b) that is coupled to the complementary region 110 of the primer nucleic acid molecule. The non-complementary overhang directs the linear primer NA molecule into and/or through the nanopore. The initial portion of the growing strand 130a flows through the nanopore and at least a portion of the subsequent portion of the growing strand 130b may flow through the nanopore, and signal or change thereof from the nanopore sensor (e.g., impedance or impedance change of the nanopore sensor) can be detected by the nanopore sensor, and the resulting data can be utilized to identify the target nucleic acid molecule.

In some cases, an impedance or impedance change may be detected by applying a constant voltage (e.g., a sinusoidal voltage perturbation) while measuring a current (e.g., a change in the current). An impedance value (Z) may be measured by a value of the applied voltage (V) divided by a value of the measured current (I). The number of impedance measurements taken may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more. The number of impedance measurements taken may be at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, or at most 1.

There can be a time lag between a first impedance measurement and a second impedance measurement. The first and second impedance measurements can be measurements of impedance or change thereof from incorporation of two different nucleobases into the growing strand, respectively. Alternatively, the first and second impedance measurements can be measurements of impedance or change thereof from incorporation of a same nucleobase into the growing strand. For example, a plurality of impedance measurements can be performed for incorporation of the same nucleobase into the growing strand (e.g., at least 2, 3, 4, 5, 6, 7, 8, 19, 10, 15, 20, 25, 30, 40, 50, or more impedance measurements). In some cases, a time lag between the first impedance measurement and the second impedance measurement can be at least about 0.1 milliseconds, at least about 0.2 milliseconds, at least about 0.3 milliseconds, at least about 0.4 milliseconds, at least about 0.5 milliseconds, at least about 0.6 milliseconds, at least about 0.7 milliseconds, at least about 0.8 milliseconds, at least about 0.9 milliseconds, at least about 1 milliseconds, at least about 1.5 milliseconds, at least about 2 milliseconds, at least about 3 milliseconds, at least about 4 milliseconds, at least about 5 milliseconds, at least about 6 milliseconds, at least about 7 milliseconds, at least about 8 milliseconds, at least about 9 milliseconds, at least about 10 milliseconds, or more. In some cases, a time lag between the first impedance measurement and the second impedance measurement can be at most about 10 milliseconds, at most about 9 milliseconds, at most about 8 milliseconds, at most about 7 milliseconds, at most about 6 milliseconds, at most about 5 milliseconds, at most about 4 milliseconds, at most about 3 milliseconds, at most about 2 milliseconds, at most about 1.5 milliseconds, at most about 1 milliseconds, at most about 0.9 milliseconds, at most about 0.8 milliseconds, at most about 0.7 milliseconds, at most about 0.6 milliseconds, at most about 0.5 milliseconds, at most about 0.4 milliseconds, at most about 0.3 milliseconds, at most about 0.2 milliseconds, at most about 0.1 milliseconds, or less.

The detected impedance or impedance change, e.g., between the sensing electrode and the reference electrode, may be at least about 1 micro-ohm, 2 micro-ohms, 3 micro-ohms, 4 micro-ohms, 5 micro-ohms, 6 micro-ohms, 7 micro-ohms, 8 micro-ohms, 9 micro-ohms, 10 micro-ohms, 20 micro-ohms, 30 micro-ohms, 40 micro-ohms, 50 micro-ohms, 60 micro-ohms, 70 micro-ohms, 80 micro-ohms, 90 micro-ohms, 100 micro-ohms, 200 micro-ohms, 300 micro-ohms, 400 micro-ohms, 500 micro-ohms, 600 micro-ohms, 700 micro-ohms, 800 micro-ohms, 900 micro-ohms, 1 milli-ohm, 2 milli-ohms, 3 milli-ohms, 4 milli-ohms, 5 milli-ohms, 6 milli-ohms, 7 milli-ohms, 8 milli-ohms, 9 milli-ohms, 10 milli-ohms, 20 milli-ohms, 30 milli-ohms, 40 milli-ohms, 50 milli-ohms, 60 milli-ohms, 70 milli-ohms, 80 milli-ohms, 90 milli-ohms, 100 milli-ohms, 200 milli-ohms, 300 milli-ohms, 400 milli-ohms, 500 milli-ohms, 600 milli-ohms, 700 milli-ohms, 800 milli-ohms, 900 milli-ohms, 1 ohm, 2 ohms, 3 ohms, 4 ohms, 5 ohms, 6 ohms, 7 ohms, 8 ohms, 9 ohms, 10 ohms, 20 ohms, 30 ohms, 40 ohms, 50 ohms, 60 ohms, 70 ohms, 80 ohms, 90 ohms, 100 ohms, 200 ohms, 300 ohms, 400 ohms, 500 ohms, 600 ohms, 700 ohms, 800 ohms, 900 ohms, 1 kilo-ohm, 2 kilo-ohms, 3 kilo-ohms, 4 kilo-ohms, 5 kilo-ohms, 6 kilo-ohms, 7 kilo-ohms, 8 kilo-ohms, 9 kilo-ohms, 10 kilo-ohms, 20 kilo-ohms, 30 kilo-ohms, 40 kilo-ohms, 50 kilo-ohms, 60 kilo-ohms, 70 kilo-ohms, 80 kilo-ohms, 90 kilo-ohms, 100 kilo-ohms, 200 kilo-ohms, 300 kilo-ohms, 400 kilo-ohms, 500 kilo-ohms, 600 kilo-ohms, 700 kilo-ohms, 800 kilo-ohms, 900 kilo-ohms, 1,000 kilo-ohms, 10,000 kilo-ohms, 100,000 kilo-ohms, 1 giga-ohms, 10 giga-ohms, 50 giga-ohms, 100 giga-ohms, or more. The detected impedance or impedance change, e.g., between the sensing electrode and the reference electrode, may be at most about 100 giga-ohms, 50 giga-ohms, 10 giga-ohms, 1 giga-ohms, 100,000 kilo-ohms, 10,000 kilo-ohms, 1,000 kilo-ohms, 900 kilo-ohms, 800 kilo-ohms, 700 kilo-ohms, 600 kilo-ohms, 500 kilo-ohms, 400 kilo-ohms, 300 kilo-ohms, 200 kilo-ohms, 100 kilo-ohms, 90 kilo-ohms, 80 kilo-ohms, 70 kilo-ohms, 60 kilo-ohms, 50 kilo-ohms, 40 kilo-ohms, 30 kilo-ohms, 20 kilo-ohms, 10 kilo-ohms, 9 kilo-ohms, 8 kilo-ohms, 7 kilo-ohms, 6 kilo-ohms, 5 kilo-ohms, 4 kilo-ohms, 3 kilo-ohms, 2 kilo-ohms, 1 kilo-ohm, 900 ohms, 800 ohms, 700 ohms, 600 ohms, 500 ohms, 400 ohms, 300 ohms, 200 ohms, 100 ohms, 90 ohms, 80 ohms, 70 ohms, 60 ohms, 50 ohms, 40 ohms, 30 ohms, 20 ohms, 10 ohms, 9 ohms, 8 ohms, 7 ohms, 6 ohms, 5 ohms, 4 ohms, 3 ohms, 2 ohms, 1 ohm, 900 milli-ohms, 800 milli-ohms, 700 milli-ohms, 600 milli-ohms, 500 milli-ohms, 400 milli-ohms, 300 milli-ohms, 200 milli-ohms, 100 milli-ohms, 90 milli-ohms, 80 milli-ohms, 70 milli-ohms, 60 milli-ohms, 50 milli-ohms, 40 milli-ohms, 30 milli-ohms, 20 milli-ohms, 10 milli-ohms, 9 milli-ohms, 8 milli-ohms, 7 milli-ohms, 6 milli-ohms, 5 milli-ohms, 4 milli-ohms, 3 milli-ohms, 2 milli-ohms, 1 milli-ohm, 900 micro-ohms, 800 micro-ohms, 700 micro-ohms, 600 micro-ohms, 500 micro-ohms, 400 micro-ohms, 300 micro-ohms, 200 micro-ohms, 100 micro-ohms, 90 micro-ohms, 80 micro-ohms, 70 micro-ohms, 60 micro-ohms, 50 micro-ohms, 40 micro-ohms, 30 micro-ohms, 20 micro-ohms, 10 micro-ohms, 9 micro-ohms, 8 micro-ohms, 7 micro-ohms, 6 micro-ohms, 5 micro-ohms, 4 micro-ohms, 3 micro-ohms, 2 micro-ohms, 1 micro-ohm, or less.

The detected impedance or impedance change, e.g., between the sensing electrode and the reference electrode, may be a measurement (e.g., a single measurement, a plurality of measurements to yield an average value of the plurality of measurements) taken over a period of at least about 1 nanosecond, 2 nanoseconds, 3 nanoseconds, 4 nanoseconds, 5 nanoseconds, 6 nanoseconds, 7 nanoseconds, 8 nanoseconds, 9 nanoseconds, 10 nanoseconds, 20 nanoseconds, 30 nanoseconds, 40 nanoseconds, 50 nanoseconds, 60 nanoseconds, 70 nanoseconds, 80 nanoseconds, 90 nanoseconds, 100 nanoseconds, 200 nanoseconds, 300 nanoseconds, 400 nanoseconds, 500 nanoseconds, 600 nanoseconds, 700 nanoseconds, 800 nanoseconds, 900 nanoseconds, 1 microsecond, 2 microseconds, 3 microseconds, 4 microseconds, 5 microseconds, 6 microseconds, 7 microseconds, 8 microseconds, 9 microseconds, 10 microseconds, 20 microseconds, 30 microseconds, 40 microseconds, 50 microseconds, 60 microseconds, 70 microseconds, 80 microseconds, 90 microseconds, 100 microseconds, 200 microseconds, 300 microseconds, 400 microseconds, 500 microseconds, 600 microseconds, 700 microseconds, 800 microseconds, 900 microseconds, 1 millisecond, 2 milliseconds, 3 milliseconds, 4 milliseconds, 5 milliseconds, 6 milliseconds, 7 milliseconds, 8 milliseconds, 9 milliseconds, 10 milliseconds, 20 milliseconds, 30 milliseconds, 40 milliseconds, 50 milliseconds, 60 milliseconds, 70 milliseconds, 80 milliseconds, 90 milliseconds, 100 milliseconds, 200 milliseconds, 300 milliseconds, 400 milliseconds, 500 milliseconds, 600 milliseconds, 700 milliseconds, 800 milliseconds, 900 milliseconds, 1 second, 2 seconds, 3 seconds, 4 seconds, 5 seconds, 6 seconds, 7 seconds, 8 seconds, 9 seconds, 10 seconds, or more. The detected impedance or impedance change, e.g., between the sensing electrode and the reference electrode, may be a measurement (e.g., a single measurement, a plurality of measurements to yield an average value of the plurality of measurements) taken over a period of at most about 10 seconds, 9 seconds, 8 seconds, 7 seconds, 6 seconds, 5 seconds, 4 seconds, 3 seconds, 2 seconds, 1 second, 900 milliseconds, 800 milliseconds, 700 milliseconds, 600 milliseconds, 500 milliseconds, 400 milliseconds, 300 milliseconds, 200 milliseconds, 100 milliseconds, 90 milliseconds, 80 milliseconds, 70 milliseconds, 60 milliseconds, 50 milliseconds, 40 milliseconds, 30 milliseconds, 20 milliseconds, 10 milliseconds, 9 milliseconds, 8 milliseconds, 7 milliseconds, 6 milliseconds, 5 milliseconds, 4 milliseconds, 3 milliseconds, 2 milliseconds, 1 millisecond, 900 microseconds, 800 microseconds, 700 microseconds, 600 microseconds, 500 microseconds, 400 microseconds, 300 microseconds, 200 microseconds, 100 microseconds, 90 microseconds, 80 microseconds, 70 microseconds, 60 microseconds, 50 microseconds, 40 microseconds, 30 microseconds, 20 microseconds, 10 microseconds, 9 microseconds, 8 microseconds, 7 microseconds, 6 microseconds, 5 microseconds, 4 microseconds, 3 microseconds, 2 microseconds, 1 microsecond, 900 nanoseconds, 800 nanoseconds, 700 nanoseconds, 600 nanoseconds, 500 nanoseconds, 400 nanoseconds, 300 nanoseconds, 200 nanoseconds, 100 nanoseconds, 90 nanoseconds, 80 nanoseconds, 70 nanoseconds, 60 nanoseconds, 50 nanoseconds, 40 nanoseconds, 30 nanoseconds, 20 nanoseconds, 10 nanoseconds, 9 nanoseconds, 8 nanoseconds, 7 nanoseconds, 6 nanoseconds, 5 nanoseconds, 4 nanoseconds, 3 nanoseconds, 2 nanoseconds, 1 nanosecond, or less.

As provided herein, the present disclosure provides a system for analyzing or identifying a target molecule. The system may have the necessary components to be configured to practice or implement any of the methods disclosed herein. The system may comprise a sensor comprising a sensing electrode and a reference electrode in electrical communication with one another. The sensor may comprise a dielectric material coupled to the sensing electrode and covering a first portion of a surface of the sensing electrode. The sensor may comprise a conducting material coupled to the sensing electrode and covering a second portion of the surface of the sensing electrode. The sensor may comprise a binding unit coupled to the conducting material, wherein the binding unit is configured to bind the target molecule. The sensor may be configured to detect one or more signals indicative of an impedance or impedance change in the sensor when at least a portion of the target molecule is bound by the binding unit. The one or more signals may be usable to analyze or identify the target molecule. The conducting material may be a bond (e.g., a chemical bond) or may comprise a linking unit (e.g., a nanorod, a peptide, a small molecule, etc.) of any desired dimension (e.g., length, cross-sectional diameter or area, volume, etc.). In an alternative aspect, the binding unit may be directly coupled to the sensing electrode. Yet in a different aspect, the binding unit may be coupled to at least a portion of the dielectric material that is coupled to the sensing electrode.

The one or more signals may be indicative of (i) electrical resistance or a change thereof in the sensor, (ii) electrical capacitance or a change thereof in the sensor, or (ii) an electrical inductance or a change thereof in the sensor. The one or more signals may be indicative of at least two of: (i) electrical resistance or a change thereof in the sensor, (ii) electrical capacitance or a change thereof in the sensor, and (ii) an electrical inductance or a change thereof in the sensor. The one or more signals may be indicative of (i) electrical resistance or a change thereof in the sensor, (ii) electrical capacitance or a change thereof in the sensor, or (ii) an electrical inductance and a change thereof in the sensor.

The one or more signals may be current or voltage. The one or more signals may be current and voltage. The one or more signals may not be tunneling current.

The first portion of the sensing electrode that is covered by the dielectric material may be at least 50 percent (%) of the surface of the sensing electrode. In some cases, the first portion of the sensing electrode may be at least 50%, 55%, 60%, 65%, 70%, 75%, 80%, 85%, 90%, 95%, 99%, or more of the surface of the sensing electrode. In some cases, the first portion of the sensing electrode may be at most 100%, 95%, 90%, 85%, 80%, 75%, 70%, 65%, 60%, 55%, 50%, or less of the surface of the sensing electrode.

The second portion of the sensing electrode may be at most 50% of the surface of the sensing electrode. In some cases, the second portion of the sensing electrode may be at most 50%, 45%, 0%, 35%, 0%, 25%, 20%, 15%, 10%, 9%, 8%, 7%, 6%, 5%, 4%, 3%, 2%, 1%, or less of the surface of the sensing electrode. In some cases, the second portion of the sensing electrode may be at least 1%, 2%, 3%, 4%, 5%, %, 7%, 8%, 9%, 10%, 15%, 20%, 25%, 30%, 35%, 40%, 45%, 50%, or more of the surface of the sensing electrode.

An average cross-sectional dimension (or a surface area that is coupled to the dielectric material and the conducting material) of the sensing electrode may be no more than 100-fold greater than an average size of the target molecule. In some cases, the average cross-sectional dimension of the sensing electrode may be at most 100-fold, 90-fold, 80-fold, 70-fold, 60-fold, 50-fold, 40-fold, 30-fold, 25-fold, 20-fold, 15-fold, 10-fold, 9-fold, 8-fold, 7-fold, 6-fold, 5-fold, 4-fold, 3-fold, 2-fold, 1-fold, 0.5-fold, or 0.1-fold greater than the average size of the target molecule. In some cases, the average cross-sectional dimension of the sensing electrode may be at least 0.1-fold, 0.5-fold, 1-fold, 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, or 100-fold greater than the average size of the target molecule. In some cases, the average cross-sectional dimension of the sensing electrode may be no more than at least 0.1 nanometers (nm), 0.5 nm, 1 nm, 2 nm, 3 nm, 4 nm, 5 nm, 10 nm, 50 nm, 100 nm, 500 nm, 1,000 nm, 5,000 nm, 10,000 nm, or more than the average size of the target molecule. In some cases, the average cross-sectional dimension of the sensing electrode may be at most 10,000 nm, 5,000 nm, 1,000 nm, 500 nm, 100 nm, 50 nm, 10 nm, 5 nm, 4 nm, 3 nm, 2 nm, 1 nm, 0.5 nm, 0.1 nm, or less than the average size of the target molecule.

Alternatively, the average cross-sectional dimension of the sensing electrode may be smaller than the average size of the target molecule. In some cases, the average cross-sectional dimension of the sensing electrode may be at least 1%, 2%, 3%, 4%, 5%, 6%, 7%, 8%, 9%, 10%, 15%, 20%, 25%, 30%, 40%, or 50% smaller than the average size of the target molecule. In some cases, the average cross-sectional dimension of the sensing electrode may be at most 50%, 40%, 30%, 20%, 15%, 10%, 9%, 8%, 7%, 6%, 5%, 4%, 3%, 2%, or 1% smaller than the average size of the target molecule.

The area of the second portion of the surface of the sensing electrode may be no more than 500 Angstrom squared (Å²), 100 Å², 50 Å², 10 Å², 9 Å², 8 Å², 7 Å², 6 Å², 5 Å², 4 Å², 3 Å², 2 Å², 1 Å², or less. In some cases, a cross-sectional dimension or the diameter of the second portion of the surface of the sensing electrode may be approximately equal to a diameter (e.g., twice that of the Van der Waals radius) of an atom of the conducting material.

The dielectric material may be a solid layer (e.g., a solid metal or semi-conducting material) or a self-assembled monolayer (SAM).

The conducting material may be a single molecule (e.g., a single conducting polymeric chain). In some cases, one or more features of atomic force microscopy (AFM) (e.g., a piezoelectric cantilever probe of the AFM) to couple the single molecule to a specific (or random) position within the surface of the sensing electrode. Alternatively, the conducting material may be a plurality of molecules (e.g., a plurality of identical and/or different conducting polymeric chains). The conducting material may be comprised of at least 1, 5, 10, 50, 100, 500, 1,000, 5,000, 10,000, 50,000, 100,000, or more molecules. The conducting material may be comprised of at most 100,000, 50,000, 10,000, 5,000, 1,000, 500, 100, 50, 10, 5, or 1 molecule.

The sensor of the present disclosure may be configured to detect more signals indicative of the impedance or impedance change, e.g., between the sensing electrode and the reference electrode, when a distance between (i) at least a portion of a target molecule (and/or a tag coupled to the target molecule) and (ii) the sensing electrode is at least about 0.1 nm, 0.5 nm, 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The sensor as disclosed herein may be configured to detect more signals indicative of the impedance or impedance change, e.g., between the sensing electrode and the reference electrode, when a distance between (i) at least a portion of a target molecule (and/or a tag coupled to the target molecule) and (ii) the sensing electrode is at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 9 nm, 8 nm, 7 nm, 6 nm, 5 nm, 4 nm, 3 nm, 2 nm, 1 nm, 0.5 nm, 0.1 nm, or less.

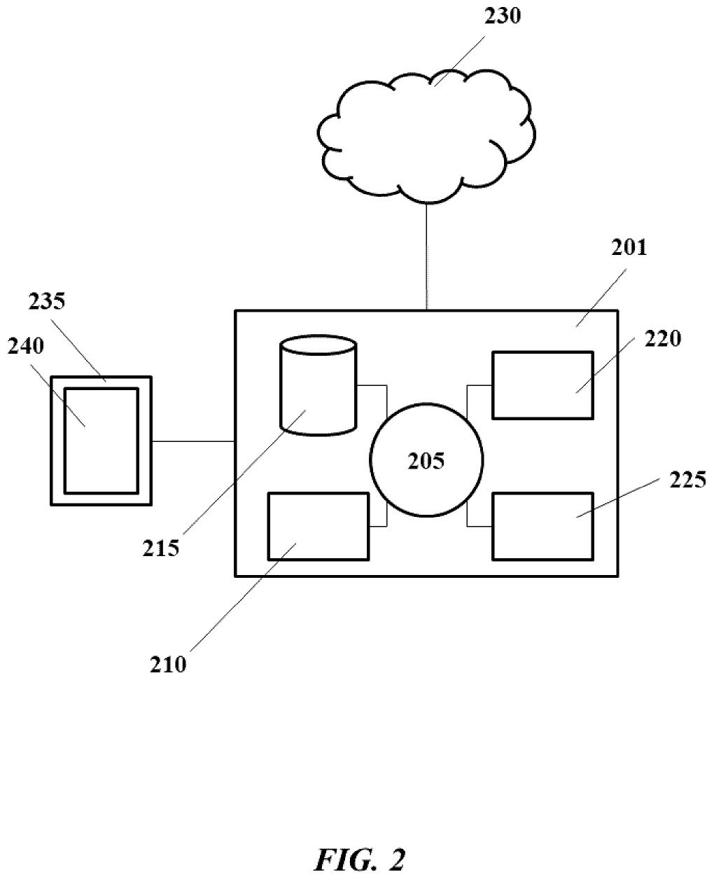

The sensor of the present disclosure may be configured to detect more signals indicative of the impedance or impedance change, e.g., between the sensing electrode and the reference electrode, when a target molecule (and/or a tag coupled to the target molecule) is within a predetermined space (e.g., the predetermined volume 217 as shown in, for instance, FIG. 2A) that is near or adjacent to the sensing electrode. The predetermined space may be characterized by having a volume of at least about 0.1 nm², 0.5 nm², 1 nm, 2 nm², 3 nm², 4 nm², 5 nm², 6 nm², 7 nm², 8 nm², 9 nm², 10 nm², 20 nm², 30 nm², 40 nm², 50 nm², 60 nm², 70 nm², 80 nm², 90 nm², 100 nm², 200 nm², 300 nm², 400 nm², 500 nm², 600 nm², 700 nm², 800 nm², 900 nm², 1 μm², 2 μm², 3 μm², 4 μm², 5 μm², 6 μm², 7 μm², 8 μm², 9 μm², 10 μm², 20 μm², 30 μm², 40 μm², 50 μm², 60 μm², 70 μm², 80 μm², 90 μm², 100 μm², 200 μm², 300 μm², 400 μm², 500 μm², 600 μm², 700 μm², 800 μm², 900 μm², 1,000 μm², or more. The predetermined space may be characterized by having a volume of at most about 1,000 μm², 900 μm², 800 μm², 700 μm², 600 μm², 500 μm², 400 μm², 300 μm², 200 μm², 100 μm², 90 μm², 80 μm², 70 μm², 60 μm², 50 μm², 40 μm², 30 μm², 20 μm², 10 μm², 9 μm², 8 μm², 7 μm², 6 μm², 5 μm², 4 μm², 3 μm², 2 μm², 1 μm², 900 nm², 800 nm², 700 nm², 600 nm², 500 nm², 400 nm², 300 nm², 200 nm², 100 nm², 90 nm², 80 nm², 70 nm², 60 nm², 50 nm², 40 nm², 30 nm², 20 nm², 10 nm², 9 nm², 8 nm², 7 nm², 6 nm², 5 nm², 4 nm², 3 nm², 2 nm², 1 nm², 0.5 nm², 0.1 nm², or less.

The sensor of the present disclosure may not require at least a portion of a target molecule (and/or a tag coupled to the target molecule) to enter and/or pass through a pore (e.g., a nanopore, such as a protein nanopore or a solid state nanopore) to detect one or more signals indicative of the impedance or impedance change. For example, the sensor may not comprise or may not be operatively coupled to a nanopore. Alternatively, at least a portion of a target molecule (and/or a tag coupled to the target molecule) may enter and/or pass through a pore of a sensor (e.g., a nanopore, such as a protein nanopore or a solid state nanopore) in order for the sensor to detect one or more signals indicative of the impedance or impedance change. For example, the sensor may comprise a nanopore.

The system may comprise at least 1, 2, 3, 4, 5, or more additional electric field generators. The system may comprise at most 5, 4, 3, 2, or 1 additional electric field generator. When comprising a plurality of additional electric field generators, the plurality of additional electric field generators may apply a plurality of electric fields that are along the same or different directions. Alternatively, the system may not comprise any additional electric field generator.

The sensor of the present disclosure may an electrical circuit (e.g., CMOS or FET circuit). The electrical circuit may be coupled to a voltage source. A constant voltage may be applied to the electrical circuit, and a change in the current may be measured. Alternatively, a change in voltage necessary to maintain a steady state current may be measured. The sensor may be in an electrolytic solution (e.g., 0.5 M Potassium Acetate and 10 mM KCl). Alternatively, the sensor may not be in an electrolytic solution. In some examples, the sensor may be in an aqueous solution or gas.

The one or more signals may be a current or voltage measured from the sensing circuit. The one or more signals may be a current and voltage measured from the sensing circuit. The signal may be a tunneling current. Alternatively, the signal may not be a tunneling current. The current may be a Faradaic current. Alternatively, the current may not be a Faradaic current. The current may be at least 1 picoamp (pA), 10 pA, 100 pA, 1 nanoamp (nA), 10 nA, 100 nA, 1 microamp (mA), 10 mA, 100 mA, or more. The current may be at most 100 mA, 10 mA, 1 mA, 100 nA, 10 nA, 1 nA, 100 pA, 10 pA, 1 pA, or less. The current may be at least in the picoamp (pA) range, tens of pA range, hundreds of pA range, nanoamp (nA) range, tens of nA range, hundreds of nA range, microamp (mA) range, tens of mA range, or higher. The current may be at most in the tens of mA range, mA range, hundreds of nA range, tens of nA range, nA range, hundreds of pA range, tens of pA range, pA range, or lower. The voltage may be at least 0.1 millivolt (mV), 0.5 mV, 1 mV, 5 mV, 10 mV, 50 mV, 100 mV, 500 mV, or more. The voltage may be at most 500 mV, 100 mV, 50 mV, 10 mV, 5 mV, 1 mV, 0.5 mV, 0.1 mV, or less. The voltage may be at least in the millivolt (mV) range, tens of mV range, hundreds of mV range, or higher. The voltage may be at most in the hundreds of mV range, tens of mV range, mV range, or lower.

In some embodiments, the sensor of the present disclosure may be provided as arrays, such as arrays present on a chip or biochip. The array of sensors may have any suitable number of any sensor of the present disclosure. The array may comprise about 10, about 20, about 50, about 100, about 200, about 400, about 600, about 800, about 1000, about 1500, about 2000, about 3000, about 4000, about 5000, about 10000, about 15000, about 20000, about 40000, about 60000, about 80000, about 100000, about 200000, about 400000, about 600000, about 800000, about 1000000, or more sensors.

In another aspect, the present disclosure provides a method for analyzing or identifying a target molecule. The method may comprise using a sensor to detect one or more signals indicative of an impedance or impedance change in the sensor when at least a portion of the target molecule is bound by at least a portion of the sensor. The method may further comprise using the one or more signals to analyze or identify the target molecule.

In another aspect, the present disclosure provides a method for analyzing or identifying a target molecule. The method may comprise providing a sensor comprising a sensing electrode and a reference electrode in electrical communication with one another. The sensor may further comprise a dielectric material coupled to the sensing electrode and covering a first portion of a surface of the sensing electrode. The sensor may further comprise a conducting material coupled to the sensing electrode and covering a second portion of the surface of the sensing electrode. The method may further comprise detecting one or more signals indicative of an impedance or impedance change in the sensor when at least a portion of the target molecule is bound by the binding moiety. The method may further comprise using the one or more signals to analyze or identify the target molecule.

In some embodiments, the sensing electrode and the reference electrode may provide a first electric field. Additionally, the method may further comprise providing an additional electric field generator. The method may further comprise using the additional electric field generator to apply a second electric field in a second direction that is approximately perpendicular to a first direction of the first electric field.

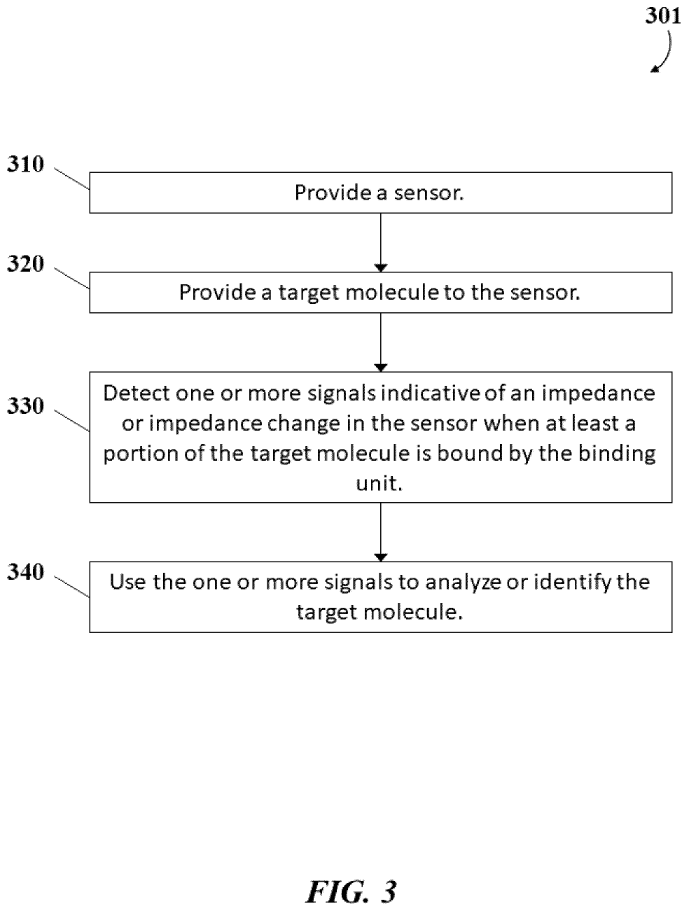

FIG. 3 shows an example process 301 of the method for analyzing or identifying a target molecule. The method comprises providing a sensor (process 310). The sensor may comprise a binding unit. The method comprises providing a target molecule to the sensor (process 320). The method comprises detecting one or more signals indicative of an impedance or impedance change in the sensor when at least a portion of the target molecule is bound by the binding unit (process 330). The method comprises using the one or more signals to analyze or identify the target molecule.

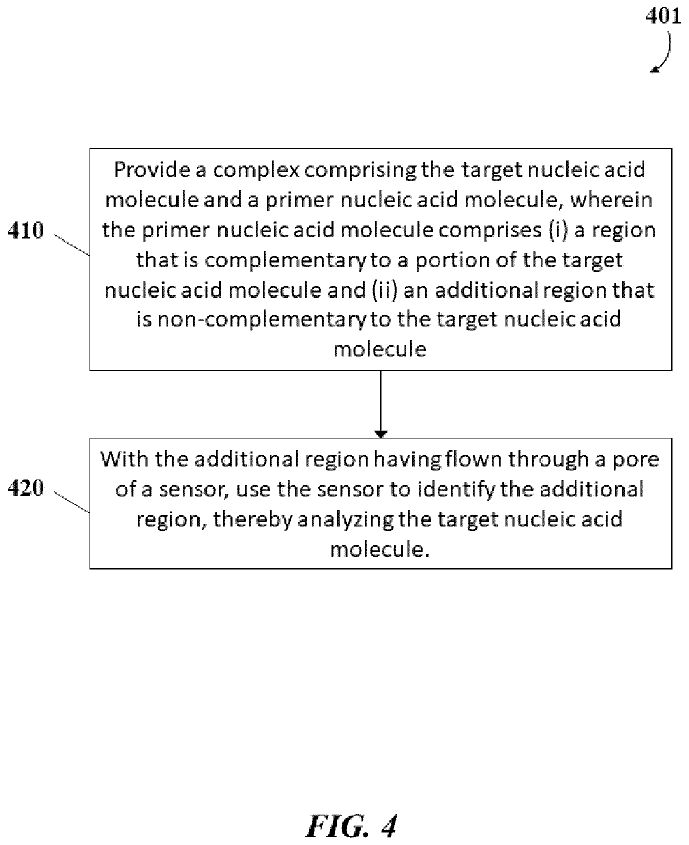

FIG. 4 shows an additional example process 401 of the method for analyzing or identifying a target nucleic acid molecule. The method comprises providing a complex comprising the target nucleic acid molecule and a primer nucleic acid molecule, wherein the primer nucleic acid molecule comprises (i) a region that is complementary to a portion of the target nucleic acid molecule and (ii) an additional region that is non-complementary to the target nucleic acid molecule (process 410). The method further comprises, with the additional region having flown through a pore of a sensor, using the sensor to identify the additional region, thereby analyzing the target nucleic acid molecule (process 420).

Alternatively, as provided herein, the one or more signals may be indicative of (i) electrical resistance or a change thereof in the sensor, (ii) electrical capacitance or a change thereof in the sensor, or (ii) an electrical inductance or a change thereof in the sensor. The one or more signals may be indicative of at least two of (i) electrical resistance or a change thereof in the sensor, (ii) electrical capacitance or a change thereof in the sensor, or (ii) an electrical inductance or a change thereof in the sensor. The one or more signals may be indicative of (i) electrical resistance or a change thereof in the sensor, (ii) electrical capacitance or a change thereof in the sensor, and (ii) an electrical inductance or a change thereof in the sensor. The one or more signals may be current or voltage. The one or more signals may be current and voltage. The one or more signals may not be a tunneling current.

### II. Sample

Samples for analysis can comprise one or more polynucleotides (e.g., a plurality of polynucleotides). A polynucleotide can be single stranded DNA, double stranded DNA, or a combination thereof. The polynucleotides can comprise genomic DNA, genomic cDNA, cell free DNA, cell free cDNA, or a combination of any of the foregoing.

A polynucleotide can include cell-free DNA, circulating tumor DNA, genomic DNA, and DNA from formalin fixed and paraffin embedded (FFPE) samples. In some examples, an extracted DNA from a FFPE sample may be damaged, and such damaged DNA may be repaired by an available FFPE DNA repair kit. A sample can comprise any suitable DNA and/or cDNA sample such as for example, urine, stool, blood, saliva, tissue, biopsy, bodily fluid, or tumor cells.

The plurality of polynucleotides can be single-stranded or double-stranded.

A polynucleotide sample can be derived from any suitable source. For example, a sample can be obtained from a patient, from an animal, from a plant, or from the environment such as, for example, a naturally occurring or artificial atmosphere, a water system, soil, an atmospheric pathogen collection system, a sub-surface sediment, groundwater, or a sewage treatment plant.

Polynucleotides from a sample may include one more different polynucleotides, such as, for example, DNA, RNA, ribosomal RNA (rRNA), transfer RNA (tRNA), micro RNA (miRNA), messenger RNA (mRNA), fragments of any of foregoing, or combinations of any of the foregoing. A sample can comprise DNA. A sample can comprise genomic DNA. A sample can comprise mitochondrial DNA, chloroplast DNA, plasmid DNA, bacterial artificial chromosomes, yeast artificial chromosomes, oligonucleotide tags, or a combination of any of the foregoing.

The polynucleotides may be single-stranded, double-stranded, or a combination thereof. A polynucleotide can be a single-stranded polynucleotide, which may or may not be in the presence of double-stranded polynucleotides.

The starting amount of polynucleotides in a sample can be, for example, less than about 50 nanograms (ng), such as less than about 45 ng, less than about 40 ng, less than about 35 ng, less than about 30 ng, less than about 25 ng, less than about 20 ng, less than about 15 ng, less than about 10 ng, less than about 5 ng, less than about 4 ng, less than about 3 ng, less than about 2 ng, less than about 1 ng, less than about 0.5 ng, less than about 0.1 ng, or less. The starting amount of polynucleotides in a sample can be, for example, more than about 0.1 ng, such as more than about 0.5 ng, more than about 1 ng, more than about 2 ng, more than about 3 ng, more than about 4 ng, more than about 5 ng, more than about 10 ng, more than about 15 ng, more than about 20 ng, more than about 25 ng, more than about 30 ng, more than about 35 ng, more than about 40 ng, more than about 45 ng, more than about 50 ng, or more. An amount of starting polynucleotides can be, for example, from about 0.1 ng to about 100 ng, from about 1 ng to about 75 ng, from about 5 ng to about 50 ng, or from about 10 ng to about 20 ng.

The polynucleotides in a sample can be single-stranded, either as obtained or by way of treatment (e.g., denaturation). Further examples of suitable polynucleotides are described herein, such as with respect to any of the various aspects of the disclosure. Polynucleotides can be subjected to subsequent steps (e.g., circularization and amplification) without an extraction step, and/or without a purification step. For example, a fluid sample may be treated to remove cells without an extraction step to produce a purified liquid sample and a cell sample, followed by isolation of the polynucleotides from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, which may correspond to dead or damaged cells. The identity of such cells may be used to characterize the cells or population of cells from which they are derived, such as in a microbial community.

A sample can be from a subject. A subject can be any suitable organism including, for example, plants, animals, fungi, protists, monerans, viruses, mitochondria, and chloroplasts. Sample polynucleotides can be isolated from a subject, such as a cell sample, tissue sample, bodily fluid sample, or organ sample or cell cultures derived from any of these, including, for example, cultured cell lines, biopsy, blood sample, cheek swab, or fluid sample containing a cell such as saliva. The subject may be an animal such as a cow, a pig, a mouse, a rat, a chicken, a cat, a dog, or a mammal, such as a human. A sample can comprise tumor cells, such as in a sample of tumor tissue from a subject.

A sample may not comprise intact cells, can be treated to remove cells, or polynucleotides are isolated without a cellular extractions step such as to isolate cell-free polynucleotides, such as cell-free DNA.

Other examples of sample sources include those from blood, urine, feces, nares, the lungs, the gut, other bodily fluids or excretions, a derivative thereof, or a combination thereof.

A sample from a single individual can be divided into multiple separate samples, such as 2, 3, 4, 5, 6, 7, 8, 9, 10, or more separate samples that are subjected to methods of the disclosure independently, such as analysis in duplicate, triplicate, quadruplicate, or more. Where a sample is from a subject, a reference sequence may also be derived from the subject, such as a consensus sequence from the sample under analysis or the sequence of polynucleotides from another sample or tissue of the same subject. For example, a blood sample may be analyzed for ctDNA mutations, and cellular DNA from another sample from the subject such as a buccal or skin sample, can be analyzed to determine a reference sequence.

Polynucleotides can be extracted from a sample, with or without extraction from cells in a sample, according to any suitable method.

A plurality of polynucleotides can comprise cell-free polynucleotides, such as cell-free DNA (cfDNA) or circulating tumor DNA (ctDNA). Cell-free DNA circulates in both healthy and diseased individuals. cfDNA from tumors (ctDNA) is not confined to any specific cancer type but appears to be a common finding across different malignancies. The free circulating DNA concentration in plasma can be lower in control subjects in comparison to that in patients having or suspected of having a condition. In an example, the free circulating DNA concentration in plasma can be, for example, from 14 ng/ml to 18 ng/mL in control subjects and from 18 ng/ml to 318 ng/mL in patients with neoplasia.

Apoptotic and necrotic cell death may contribute to cell-free circulating DNA in bodily fluids. For example, significantly increased circulating DNA levels may be observed in plasma of prostate cancer patients and other prostate diseases, such as Benign Prostate Hyperplasia and Prostatits. In addition, circulating tumor DNA may be present in fluids originating from the organs where the primary tumor occurs. In an example, breast cancer detection can be achieved in ductal lavages; colorectal cancer detection in stool; lung cancer detection in sputum, and prostate cancer detection in urine or ejaculate. Cell-free DNA may be obtained from a variety of sources. An example source may be blood samples of a subject. However, cfDNA or other fragmented DNA may be derived from a variety of other sources including, for example, urine and stool samples can be a source of cfDNA, including ctDNA.

### III. Nanopore

A sequencing system can include a reaction chamber that includes one or more nanopore devices. A nanopore device may be an individually addressable nanopore device. An individually addressable nanopore can be individually readable. An individually addressable nanopore can be individually writable. An individually addressable nanopore can be individually readable and individually writable. The system can include one or more computer processors for facilitating sample preparation and various operations of the disclosure, such as polynucleotide sequencing. The processor can be coupled to nanopore device.

A nanopore device may include a plurality of individually addressable sensing electrodes. Each sensing electrode can include a membrane adjacent to the electrode, and one or more nanopores in the membrane. A nanopore may be in a membrane such as a lipid bi-layer disposed adjacent or in sensing proximity to an electrode that is part of, or coupled to, an integrated circuit. A nanopore may be associated with an individual electrode and sensing integrated circuit or a plurality of electrodes and sensing integrated circuits. A nanopore can comprise a solid state nanopore.

Devices and systems for use in methods provided by the present disclosure may accurately detect individual nucleotide incorporation events, such as upon the incorporation of a nucleotide into a growing strand that is complementary to a template. An enzyme such as a DNA polymerase, RNA polymerase, or ligase can incorporate nucleotides to a growing polynucleotide chain. Enzymes such as polymerases can generate polynucleotide strands.

In some cases, the incorporated nucleotides (e.g., incorporated as part of the growing strand coupled to the primer nucleic acid molecule) can be detected by the nanopore as the incorporated nucleotides are flowing through the nanopore. Accordingly, there can be a time delay between (i) a time of incorporation of a nucleotide to the growing strand and (ii) sensing of the incorporated nucleotide that is flowing through the nanopore sensor. The sensing data may not be a real-time measurement of the polymerization step or incident, but rather a post-polymerization step measurement when the polymerized nucleotide(s) enter and/or flow through the nanopore. The time delay can be at least or up to about 0.01 milliseconds (ms), at least or up to about 0.02 ms, at least or up to about 0.05 ms, at least or up to about 0.1 ms, at least or up to about 0.2 ms, at least or up to about 0.5 ms, at least or up to about 1 ms, at least or up to about 2 ms, at least or up to about 5 ms, at least or up to about 10 ms, at least or up to about 20 ms, at least or up to about 40 ms, at least or up to about 100 ms, at least or up to about 200 ms, at least or up to about 500 ms, at least or up to about 1 second, at least or up to about 2 seconds, at least or up to about 5 seconds, at least or up to about 10 seconds, at least or up to about 20 seconds, at least or up to about 30 seconds, or at least or up to about 60 seconds.

In some cases, sensing as provided herein can comprise obtaining (i) a first signal reading form the sensor obtained during the incorporation of a nucleobase (e.g., tagged nucleobase or untagged nucleobase) to the growing strand and (ii) a second signal reading from the sensor obtained when the incorporated nucleobase of the growing strand enters, is in, or is flowing through the pore of the sensor. Utilizing (e.g., comparing) both the first signal and the second signal can enhance accuracy of analysis (e.g., identification) of the incorporated nucleobase thus that of the target nucleic acid molecule.

In some cases, the nucleotides to be incorporated into the growing strand may not be tagged. Alternatively, the nucleotides to be incorporated into the growing strand can be tagged.

The added nucleotide can be complimentary to the corresponding template polynucleotide strand which is hybridized to the growing strand. A nucleotide can include a tag or tag species that is coupled to any location of the nucleotide including, but not limited to a phosphate such as a γ-phosphate, sugar or nitrogenous base moiety of the nucleotide. In some cases, tags are detected while tags are associated with a polymerase during the incorporation of nucleotide tags. The tag may continue to be detected until the tag translocates through the nanopore after nucleotide incorporation and subsequent cleavage and/or release of the tag. Nucleotide incorporation events can release tags from the nucleotides which pass through a nanopore and are detected. A tag can be released by the polymerase or cleaved/released in any suitable manner including without limitation cleavage by an enzyme located near the polymerase. In this way, the incorporated base may be identified (i.e., A, C, G, T or U) because a unique tag is released from each type of nucleotide (i.e., adenine, cytosine, guanine, thymine or uracil). In nucleotide incorporation events that do not release, a tag coupled to an incorporated nucleotide is detected with the aid of a nanopore. In some examples, the tag can move through or in proximity to the nanopore and be detected with the aid of the nanopore.

Methods and systems of the disclosure can enable the detection of polynucleotide incorporation events, such as at a resolution of at least about 1, at least about 2, at least about 3, at least about 4, at least about 5, at least about 6, at least about 7, at least about 8, at least about 9, at least about 10, at least about 20, at least about 30, at least about 40, at least about 50, at least about 100, at least about 500, at least about 1000, at least about 5000, at least about 10000, at least about 50000, or at least about 100000 polynucleotide bases within a given time period. For example, a nanopore device can be used to detect individual polynucleotide incorporation events, with each event being associated with an individual nucleic acid base. In other examples, a nanopore device can be used to detect an event that is associated with a plurality of bases. For example, a signal sensed by the nanopore device can be a combined signal from at least about 2, at least about 3, at least about 4, or at least about 5 bases. Alternatively, a signal sensed by the nanopore device can be related to (or indicative of) a single base.

In certain sequencing methods, tags do not pass through the nanopore. The tags can be detected by the nanopore and exit the nanopore without passing through the nanopore such as exiting from the inverse direction from which the tag entered the nanopore. A sequencing device can be configured to actively expel the tags from the nanopore.

In certain sequencing methods tags are not released upon nucleotide incorporation events. Nucleotide incorporation events can present tags to a nanopore without releasing the tags. The tags can be detected by the nanopore without being released. The tags may be attached to the nucleotides by a linker of sufficient length to present the tag to the nanopore for detection. For example, as a tagged nucleobase that is now part of the growing strand can pass through the nanopore, and the nanopore sensor can measure a signal (e.g., impedance) or a change thereof that is indicative of the identity of presence of the tagged nucleobase in the growing strand.

Nucleotide incorporation events may be detected in real-time as they occur by a nanopore. An enzyme such as a DNA polymerase attached to or in proximity to a nanopore can facilitate the flow of a polynucleotide through or adjacent to a nanopore. A nucleotide incorporation event, or the incorporation of a plurality of nucleotides, may release or present one or more tags, which may be detected by a nanopore. Detection can occur as the tags flow through or adjacent to the nanopore, as the tags reside in the nanopore and/or as the tags are presented to the nanopore. In some cases, an enzyme attached to or in proximity to the nanopore may aid in detecting tags upon the incorporation of one or more nucleotides.

A tag can be an atom, a molecule, a collection of atoms, or a collection of molecules. A tag may provide an optical, electrochemical, magnetic, or electrostatic such as an inductive or capacitive, signature, which signature may be detected with the aid of a nanopore.

The nanopore may be formed or otherwise embedded in a membrane disposed adjacent to a sensing electrode of a sensing circuit, such as an integrated circuit. An integrated circuit may be an application specific integrated circuit (ASIC). An integrated circuit can be a field effect transistor or a complementary metal-oxide semiconductor (CMOS). A sensing circuit may be situated in a chip or other device having the nanopore, or off of the chip or device, such as in an off-chip configuration.

As a nucleic acid or tag flows through or adjacent to the nanopore, the sensing circuit detects an electrical signal associated with the nucleic acid or tag. The nucleic acid may be a subunit of a larger strand. The tag may be a byproduct of a nucleotide incorporation event or other interaction between a tagged nucleic acid and the nanopore or a species adjacent to the nanopore, such as an enzyme that cleaves a tag from a nucleic acid. The tag may remain attached to the nucleotide. A detected signal may be collected and stored in a memory location, and later used to construct a sequence of the nucleic acid. The collected signal may be processed to account for any abnormalities in the detected signal, such as errors.

Nanopores may be used to sequence polynucleotides indirectly, in some cases with electrical detection. Indirect sequencing may be any method where an incorporated nucleotide in a growing strand does not pass through the nanopore. The polynucleotide may pass within any suitable distance from and/or proximity to the nanopore, in some cases within a distance such that tags released from nucleotide incorporation events are detected in the nanopore.

Byproducts of nucleotide incorporation events may be detected by the nanopore. Nucleotide incorporation events refer to the incorporation of a nucleotide into a growing polynucleotide chain. A byproduct may be correlated with the incorporation of a given type nucleotide. Nucleotide incorporation events can be catalyzed by an enzyme, such as DNA polymerase, and use base pair interactions with a template molecule to choose amongst the available nucleotides for incorporation at each location.

A nucleic acid sample may be sequenced using tagged nucleotides or nucleotide analogs. In some examples, a method for sequencing a nucleic acid molecule comprises (a) incorporating (e.g., polymerizing) tagged nucleotides, wherein a tag associated with an individual nucleotide is released upon incorporation, and (b) detecting the released tag with the aid of a nanopore. In some instances, the method further comprises directing the tag attached to or released from an individual nucleotide through the nanopore. The released or attached tag may be directed by any suitable technique, in some cases with the aid of an enzyme (or molecular motor) and/or a voltage difference across the pore. Alternative, the released or attached tag may be directed through the nanopore without the use of an enzyme. For example, the tag may be directed by a voltage difference across the nanopore as described herein.

A tag may be detected with the aid of a nanopore device having at least one nanopore in a membrane. The tag may be associated with an individual tagged nucleotide during incorporation of the individual tagged nucleotide. A nanopore device can detect a tag associated with an individual tagged nucleotide during incorporation. The tagged nucleotides, whether incorporated into a growing nucleic acid strand or unincorporated, can be detected, determined, or differentiated for a given period of time by the nanopore device, in some cases with the aid of an electrode and/or nanopore of the nanopore device. The time period within which the nanopore device detects the tag may be shorter, in some cases substantially shorter, than the time period in which the tag and/or nucleotide coupled to the tag is held by an enzyme, such as an enzyme facilitating the incorporation of the nucleotide into a nucleic acid strand (e.g., a polymerase). A tag can be detected by the electrode a plurality of times within the time period that the incorporated tagged nucleotide is associated with the enzyme. For instance, the tag can be detected by the electrode at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 1000, at least about 10,000, at least about 100,000, or at least about 1,000,000 times within the time period that the incorporated tagged nucleotide is associated with the enzyme.

Sequencing can be accomplished using pre-loaded tags. Pre-loading a tag can comprise directing at least a portion of the tag through at least a portion of a nanopore while the tag can be attached to a nucleotide, which nucleotide has been incorporated into a nucleic acid strand (e.g., growing nucleic acid strand), is undergoing incorporation into the nucleic acid strand, or has not yet been incorporated into the nucleic acid strand but may undergo incorporation into the nucleic acid strand. Pre-loading a tag can comprise directing at least a portion of the tag through at least a portion of the nanopore before the nucleotide has been incorporated into the nucleic acid strand or while the nucleotide is being incorporated into the nucleic acid strand. Pre-loading a tag can include directing at least a portion of the tag through at least a portion of the nanopore after the nucleotide has been incorporated into the nucleic acid strand.

A tag associated with an individual nucleotide can be detected by a nanopore without being released from the nucleotide upon incorporation. Tags can be detected without being released from incorporated nucleotides during synthesis of a nucleic acid strand that is complementary to a target strand. The tags can be attached to the nucleotides with a linker such that the tag is presented to the nanopore (e.g., the tag hangs down into or otherwise extend through at least a portion of the nanopore). The length of the linker may be sufficiently long so as to permit the tag to extend to or through at least a portion of the nanopore. In some instances, the tag is presented to (i.e., moved into) the nanopore by a voltage difference. Other ways to present the tag into the pore may also be suitable (e.g., use of enzymes, magnets, electric fields, pressure differential). In some instances, no active force is applied to the tag (i.e., the tag diffuses into the nanopore).

A chip for sequencing a nucleic acid sample can comprise a plurality of individually addressable nanopores. An individually addressable nanopore of the plurality can contain at least one nanopore formed in a membrane disposed adjacent to an integrated circuit. Each individually addressable nanopore can be capable of detecting a tag associated with an individual nucleotide. The nucleotide can be incorporated (e.g., polymerized) and the tag may not be released from the nucleotide upon incorporation.

Tags can be presented to the nanopore upon nucleotide incorporation events and are released from the nucleotide. The released tags can go through the nanopore. The tags do not pass through the nanopore in some instances. A tag that has been released upon a nucleotide incorporation event is distinguished from a tag that may flow through the nanopore but has not been released upon a nucleotide incorporation event at least in part by the dwell time in the nanopore. In some cases, tags that dwell in the nanopore for at least 100 milliseconds (ms) are released upon nucleotide incorporation events and tags that dwell in the nanopore for less than 100 ms are not released upon nucleotide incorporation events. Tags may be captured and/or guided through the nanopore by a second enzyme or protein (e.g., a nucleic acid binding protein). The second enzyme may cleave a tag upon (e.g., during or after) nucleotide incorporation. A linker between the tag and the nucleotide may be cleaved.

A tag that is coupled to an incorporated nucleotide is distinguished from a tag associated with a nucleotide that has not been incorporated into a growing complementary strand based on the residence time of the tag in the nanopore or a signal detected from the unincorporated nucleotide with the aid of the nanopore. An unincorporated nucleotide may generate a signal (e.g., voltage difference, current) that is detectable for a time period between 1 nanosecond (ns) and 100 ms, or between 1 ns and 50 ms, whereas an incorporated nucleotide may generate a signal with a lifetime between 50 ms and 500 ms, or 100 ms and 200 ms. An unincorporated nucleotide may generate a signal that is detectable for a time period between 1 ns and 10 ms, or 1 ns and 1 ms. An unincorporated tag is detectable by a nanopore for a time period (average) that is longer than the time period in which an incorporated tag is detectable by the nanopore.

Incorporated nucleic acids can be detected by and/or are detectable by the nanopore for a shorter period of time than an un-incorporated nucleotide. Alternatively, incorporated nucleic acids can be detected by and/or are detectable by the nanopore for a longer period of time than an un-incorporated nucleotide. The difference and/or ratio between these times can be used to determine whether a nucleotide detected by the nanopore is incorporated or not, as described herein.

The detection period can be based on the free-flow of the nucleotide through the nanopore; an unincorporated nucleotide may dwell at or in proximity to the nanopore for a time period between 1 nanosecond (ns) and 100 ms, or between 1 ns and 50 ms, whereas an incorporated nucleotide may dwell at or in proximity to the nanopore for a time between 50 ms and 500 ms, or 100 ms and 200 ms. The time periods can vary based on processing conditions; however, an incorporated nucleotide may have a dwell time that is greater than that of an unincorporated nucleotide.

A tag or tag species can include a detectable atom or molecule, or a plurality of detectable atoms or molecules. A tag can include a one or more adenine, guanine, cytosine, thymine, uracil, or a derivative thereof linked to any position including a phosphate group, sugar or a nitrogenous base of a nucleic acid molecule. A tag can include one or more adenine, guanine, cytosine, thymine, uracil, or a derivative thereof covalently linked to a phosphate group of a nucleic acid base.

A tag can have a length of at least about 0.1 nanometers (nm), at least about 1 nm, at least about 2 nm, at least about 3 nm, at least about 4, at least about nm, at least about 5 nm, at least about 6 nm, at least about 7 nm, at least about 8 nm, at least about 9 nm, at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm, at least about 500 nm, or at least about 1000 nm.

A tag can include a tail of repeating subunits, such as a plurality of adenine, guanine, cytosine, thymine, uracil, or a derivative thereof. For example, a tag can include a tail portion having at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 1000, at least about 10,000, or at least about or 100,000 subunits of adenine, guanine, cytosine, thymine, uracil, or a derivative thereof. The subunits can be linked to one another, and at a terminal end linked to a phosphate group of the nucleic acid. Other examples of tag portions include any polymeric material, such as polyethylene glycol (PEG), polysulfonates, amino acids, or any completely or partially positively charged, negatively charged, or un-charged polymer. A tag as disclosed herein can be a label, as provided herein.

### IV. Polymerase

A DNA polymerase can be bound to the 3′ end of a nicked strand of the polynucleotide at the nicking site. DNA sequencing can be accomplished by using an enzyme such as a DNA polymerize to amplify and transcribe a polynucleotide in proximity to a nanopore and tagged nucleotides. Sequencing methods can involve incorporating or polymerizing tagged nucleotides using a polymerase such as a DNA polymerase, or transcriptase. The polymerase can be mutated to allow it to accept tagged nucleotides. The polymerase can also be mutated to increase the time for which the tag is detected by the nanopore.

A sequencing enzyme can be, for example, any suitable enzyme that creates a polynucleotide strand by phosphate linkage of nucleotides. The DNA polymerase can be, for example, a 9° Nm™ polymerase or a variant thereof, an E. Coli DNA polymerase I, a Bacteriophage T4 DNA polymerase, a Sequenase, a Taq DNA polymerase, a 9°Nm™ polymerase (exo-) A485L/Y409V, a $29 DNA Polymerase, a Bst DNA polymerase, or variants, mutants, or homologs of any of the foregoing. A homolog can have any suitable percentage homology such as, for example, at least about 30%, at least about 40%, at least about 50%, at least about 60%, at least about 70%, at least about 80%, at least about 90%, or at least about 95% sequence identity.

In some examples, for nanopore sequencing, a polymerization enzyme can be attached to or situated in proximity to a nanopore. Suitable methods for attaching the polymerization enzyme to a nanopore include cross-linking the enzyme to the nanopore or in proximity to the nanopore such as via the formation of intra-molecular disulfide bonds. The nanopore and the enzyme may also be a fusion such as an encoded by a single polypeptide chain. Methods for producing fusion proteins may include fusing the coding sequence for the enzyme in frame and adjacent to the coding sequence for the nanopore and expressing this fusion sequence from a single promoter. A polymerization enzyme can be attached or coupled to a nanopore using molecular staples or protein fingers. A polymerization enzyme can be attached to a nanopore via an intermediate molecule, such as for example biotin conjugated to both the enzyme and the nanopore with streptavidin tetramers linked to both biotins. The intermediate molecule can be referred to as a linker.

The sequencing enzyme can also be attached to a nanopore with an antibody. Proteins that form a covalent bond between each other can be used to attach a polymerase to a nanopore. Phosphatase enzymes or an enzyme that cleaves a tag from a nucleotide can also be attached to the nanopore.

The polymerase can be mutated to facilitate and/or to improve the efficiency of the mutated polymerase for incorporation of tagged nucleotides into a growing polynucleotide relative to the non-mutated polymerase. The polymerase can be mutated to improve entry of the nucleotide analog such as a tagged nucleotide, into the active site region of the polymerase and/or mutated for coordinating with the nucleotide analogs in the active region.

Other mutations such as amino acid substitutions, insertions, deletions, and/or exogenous features to a polymerize can result in enhanced metal ion coordination, reduced exonuclease activity, reduced reaction rates at one or more steps of the polymerase kinetic cycle, decreased branching fraction, altered cofactor selectivity, increased yield, increased thermostability, increased accuracy, increased speed, increased read length, increased salt tolerance relative to the non-mutated polymerase.

A suitable polymerase can have a kinetic rate profile that is suitable for detection of the tags by a nanopore. The rate profile generally refers to the overall rate of nucleotide incorporation and/or a rate of any step of nucleotide incorporation such as nucleotide addition, enzymatic isomerization such as to or from a closed state, cofactor binding or release, product release, incorporation of polynucleotide into the growing polynucleotide, or translocation.

A polymerase can be adapted to permit the detection of sequencing events. The rate profile of a polymerase can be such that a tag is loaded into (and/or detected by) the nanopore for an average of 0.1 milliseconds (ms), 1 ms, 5 ms 10 ms, 20 ms, 30 ms, 40 ms, 50 ms, 60 ms, 80 ms, 100 ms, 120 ms, 140 ms, 160 ms, 180 ms, 200 ms, 220 ms, 240 ms, 260 ms, 280 ms, 300 ms, 400 ms, 500 ms, 600 ms, 800 ms, or 1000 ms. For example, the rate profile of a polymerase can be such that a tag is loaded into and/or detected by the nanopore for an average of at least 5 ms, at least 10 ms, at least 20 ms, at least 30 ms, at least 40 ms, at least 50 ms, at least 60 ms, at least 80 ms, at least 100 ms, at least 120 ms, at least 140 ms, at least 160 ms, at least 180 ms, at least 200 ms, at least 220 ms, at least 240 ms, at least 260 ms, at least 280 ms, at least 300 ms, at least 400 ms, at least 500 ms, at least 600 ms, at least 800 ms, or at least 1000 ms. A tag can be detected by the nanopore for an average between 80 ms and 260 ms, between 100 ms and 200 ms, or between 100 ms and 150 ms.

A nanopore/polymerase complex can be configured to permit the detection of one or more events associated with amplification and transcription of the circular polynucleotide. The one or more events may be kinetically observable and/or non-kinetically observable such as a nucleotide migrating through a nanopore without coming in contact with a polymerase.

In some cases, the polymerase reaction exhibits two kinetic steps which proceed from an intermediate in which a nucleotide or a polyphosphate product is bound to the polymerase enzyme, and two kinetic steps which proceed from an intermediate in which the nucleotide and the polyphosphate product are not bound to the polymerase enzyme. The two kinetic steps can include enzyme isomerization, nucleotide incorporation, and product release. In some cases, the two kinetic steps are template translocation and nucleotide binding.

A suitable polymerase can exhibit strong or enhanced strand displacement.

### V. Identification of Sequence Variants

Methods provided by the present disclosure can be used to identify sequence variants in a polynucleotide sample. A sequence difference between sequencing reads and a reference sequence is referred to as a genuine sequence variant if the sequence difference occurs in at least two different polynucleotides, e.g., two different circular polynucleotides, which can be distinguished as a result of having different junctions. Because the position and type of a sequence variant that are the result of amplification or sequencing errors are unlikely to be duplicated exactly on two different polynucleotides comprising the same target sequence, including this validation parameter can reduce the background of erroneous sequence variants, with a concurrent increase in the sensitivity and accuracy of detecting actual sequence variation in a sample. A sequence variant can have a frequency less than less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.04%, less than 0.03%, less than 0.02%, less than 0.01%, less than 0.005%, less than 0.001%, or lower is sufficiently above background to permit an accurate identification. A sequence variant can occur with a frequency of less than 0.1%. The frequency of a sequence variant can be sufficiently above background when such frequency is statistically significantly above the background error rate, for example, with a p-value less than 0.05, 0.01, 0.001, or 0.0001. The frequency of a sequence variant can be sufficiently above background when the frequency is at least 2-fold, 3-fold, 4-fold, 5-fold, 6-fold, 7-fold, 8-fold, 9-fold, 10-fold, 25-fold, 50-fold, 100-fold, or more above the background error rate. The background error rate for accurately determining the sequence at a given position can be less than 1%, 0.5%, 0.1%, 0.05%, 0.01%, 0.005%, 0.001%, or 0.0005%.

Identifying a sequence variant can comprise optimally aligning one or more sequencing reads with a reference sequence to identify differences between the two, as well as to identify junctions. Alignment can involve placing one sequence along another sequence, iteratively introducing gaps along each sequence, scoring how well the two sequences match, and repeating for various positions along the reference. The best-scoring match is deemed to be the alignment and represents an inference about the degree of relationship between the sequences.

A reference sequence to which sequencing reads are compared is a reference genome, such as the genome of a member of the same species as the subject. A reference genome may be complete or incomplete. A reference genome can consist only of regions containing target polynucleotides, such as from a reference genome or from a consensus generated from sequencing reads under analysis. A reference sequence can comprise or can consist of sequences of polynucleotides of one or more organisms, such as sequences from one or more bacteria, archaea, viruses, protists, fungi, or other organism. A reference sequence can consist of only a portion of a reference genome, such as regions corresponding to one or more target sequences under analysis. For example, for detection of a pathogen, a reference genome can be the entire genome of the pathogen, or a portion thereof useful in identification, such as of a particular strain or serotype. A sequencing read can be aligned to multiple different reference sequences, such as to screen for multiple different organisms or strains.

### VI. Therapeutic Applications

Methods, systems, and compositions provided herein can be directed to one or more therapeutic applications, such as in the characterization of a patient sample and optionally diagnosis of a condition of a subject. Therapeutic applications can include informing the selection of therapies to which a patient may be most responsive and/or treatment of a subject in need of therapeutic intervention based on the results of methods provided by the present disclosure.

For example, methods provided by the present disclosure can be used to diagnose tumor presence, progression and/or metastasis of tumors, such as when the polynucleotides analyzed comprise or consist of cfDNA, ctDNA, or fragmented tumor DNA. A subject may be monitored for tumor treatment efficacy, for example, by monitoring ctDNA over time, a decrease in ctDNA can be used as an indication of treatment efficacy and increases in ctDNA can inform selection of different treatments and/or different dosages. Other uses include evaluations of organ rejection in transplant recipients such as where increases in the amount of circulating DNA corresponding to the transplant donor genome is used as an early indicator of transplant rejection, and genotyping/isotyping of pathogen infections, such as viral or bacterial infections. Detection of sequence variants in circulating fetal DNA may be used to diagnose a condition of a fetus.

Methods provided by the present disclosure can comprise diagnosing a subject based on a result of the sequencing, such as diagnosing the subject with a disease associated with a detected causal genetic variant or reporting a likelihood that the patient has or will develop such disease.

A causal genetic variant can include sequence variants associated with a particular type or stage of cancer, or of cancer having a particular characteristic such as metastatic potential, drug resistance, and/or drug responsiveness. Methods provided by the present disclosure can be used to inform therapeutic decisions, guidance and monitoring, of cancer therapies. For example, treatment efficacy can be monitored by comparing patient ctDNA samples from before, during, and after treatment with particular including molecular targeted therapies such as monoclonal drugs, chemotherapeutic drugs, radiation protocols, and combinations of any of the foregoing. For example, the ctDNA can be monitored to see if certain mutations increase or decrease, or new mutations appear, after treatment, which can allow a physician to modify a treatment in a much shorter period of time than afforded by methods of monitoring that track patient symptoms. Methods can comprise diagnosing a subject based on the results of polynucleotide sequencing, such as diagnosing the subject with a particular stage or type of cancer associated with a detected sequence variant or reporting a likelihood that the patient has or will develop such cancer.

For example, for therapies that are specifically targeted to patients on the basis of molecular markers, patients can be tested to find out if certain mutations are present in their tumor, and these mutations can be used to predict response or resistance to the therapy and guide the decision whether to use the therapy. Detecting and monitoring ctDNA during the course of treatment can be useful in guiding treatment selections.

Sequence variants associated with one or more kinds of cancer that may be used for diagnosis, prognosis, or treatment decisions. For example, suitable target sequences of oncological significance include alterations in the TP53 gene, the ALK gene, the KRAS gene, the PIK3CA gene, the BRAF gene, the EGFR gene, and the KIT gene. A target sequence the may be specifically amplified, and/or specifically analyzed for sequence variants may be all or part of a cancer-associated gene.

Methods provided by the present disclosure can be useful in discovering new, rare mutations that are associated with one or more cancer types, stages, or cancer characteristics. For example, in populations of individuals sharing a characteristic under analysis such as a particular disease, type of cancer, and/or stage of cancer, using methods provided by the present disclosure sequence variants can be identified reflecting mutations in particular genes or parts of genes. Identified sequence variants occurring with a statistically significantly greater frequency among the group of individuals sharing the characteristic than in individuals without the characteristic may be assigned a degree of association with that characteristic. The sequence variants or types of sequence variants so identified may then be used in diagnosing or treating individuals discovered to harbor them.

Additional therapeutic applications can include use in non-invasive fetal diagnostics. Fetal DNA can be found in the blood of a pregnant woman. Methods provided by the present disclosure can be used to identify sequence variants in circulating fetal DNA, and thus may be used to diagnose one or more genetic diseases in the fetus, such as those associated with one or more causal genetic variants. Examples of causal genetic variants include trisomies, cystic fibrosis, sickle-cell anemia, and Tay-Saks disease. The mother may provide a control sample and a blood sample to be used for comparison. The control sample may be any suitable tissue and can then be sequenced to provide a reference sequence. Sequences of cfDNA corresponding to fetal genomic DNA can then be identified as sequence variants relative to the maternal reference. The father may also provide a reference sample to aid in identifying fetal sequences, and sequence variants.

Different therapeutic applications can include detection of exogenous polynucleotides, including from pathogens such as bacteria, viruses, fungi, and microbes, which information may inform a treatment.

### VII. Computer Systems

The present disclosure provides computer systems that are programmed to implement one or more methods of the present disclosure. Computer systems of the present disclosure may be used to regulate various operations of the sensor, such as detecting one or more signals indicative of an impedance or impedance change in the sensor when at least a portion of a target molecule is bound by a binding moiety of the sensor.

FIG. 2 shows a computer system 201 that is programmed or otherwise configured to communicate with and regulate various aspects of sequencing of the present disclosure. The computer system 201 can communicate with, for example, one or more circuitry coupled to or comprising the sensor, and one or more devices (e.g., machines) used to prepare, treat, or keep one or more reaction mixtures for the sensing. The computer system 201 may also communicate with one or more controllers or processors of the present disclosure. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, (i) progress of the reaction mixture, (ii) progress of sequencing, and (iii) sequencing information obtained from sequencing. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, determine sequence readout of a target nucleotide, polynucleotide, peptide, polypeptide, protein, etc.

### EXAMPLES

### Example 1: Sequencing-by-Synthesis (SBS)

In some embodiments, a method as provided herein can be utilized for determining a polymer sequence information (e.g., a polynucleotide sequence information) comprises using a system (e.g., a device or an apparatus). In some cases, the polymer can be a target nucleic acid (NA) molecule (e.g., a template). In some cases, the determining can comprise direct read nanopore SBS.

(1A) In some embodiments, the methods can utilize a system (e.g., a device, an apparatus, etc.) comprising an array of pores or wells (e.g., nanopores) configures to hold fluid for sensing (e.g., sequencing). The fluid can be configured to hold the target NA molecule, a primer NA molecule as provided herein (e.g., comprising the complementary region and the non-complementary region), and/or a complex comprising the target NA molecule and the primer NA molecule.

(1B) In some embodiments, the system can comprise a membrane. The membrane can be used to cover at least a portion of the array of pores/wells, e.g., to provide engagement and/or support of the complex of (1A) to interact with one or more pores/wells of the array. The membrane can be configured to separate the pores/wells in (1A) into an upper fluid chamber (e.g., a cis chamber) and a lower fluid chamber (e.g., a trans chamber).

(1C) In some embodiments, the array of nanopores is engaged with (e.g., at least partially embedded in) the membrane of (1B).

(1D) In some embodiments, an array of additional complexes is engaged with (e.g., at least partially embedded in) the membrane of (1B). Each of the array of additional complexes comprises a nanopore, an enzyme (e.g., a polymerase), and the complex of (1A).

(1E) In some embodiments, the nanopores of (1C) or (1D) can be synthetic or biological pores.

(1F) In some embodiments, each complex of (1D) can be a template-primer complex.

(1G) In some embodiments, the complex can be bound to the enzyme, to form the additional complex, which additional complex is an enzyme-template-primer complex.

(1H) In some embodiments, the complex can be bound to the enzyme that is coupled to the nanopore, to form the additional complex, which additional complex is an enzyme-template-primer complex.

(1I) In some embodiments, in each additional complex of (1D), the enzyme can be coupled to the nanopore at a specific site of the nanopore (e.g., via hydrogen bonding, affinity binding, etc.).

(1J) In some embodiments, the coupling between the enzyme and the nanopore in (1I) can be permanent or can be broken, e.g., via an external force or treatment such as high voltage of high pH.

(1K) In some embodiments, the formation of each additional complex of (1D) can be achieved through forming a nanopore-enzyme sub-unit, and subsequently forming a nanopore-enzyme-template-primer complex by adding a template-primer sub-unit to the nanopore-enzyme sub-unit.

(1L) In some embodiments, the formation of each additional complex of (1D) can be achieved through forming an enzyme-template-primer sub-unit, and subsequently forming a nanopore-enzyme-template-primer complex by adding the enzyme-template-primer sub-unit to the nanopore.

(1M) In some embodiments, the formation of each additional complex of (1D) can be achieved through breaking a connection (e.g., a bonding) between the nanopore and the enzyme, and subsequently forming a new nanopore-enzyme-template-primer complex by adding new enzyme-template-primer sub-unit to the nanopore, thereby forming the new nanopore-enzyme-template-primer complex. In some cases, a nanopore can be engineered to comprise a binding moiety that can bind to an enzyme, and such binding via the binding moiety can be reversible. In some cases, an enzyme can be engineered to comprise a binding moiety that can bind to a nanopore, and such binding via the binding moiety can be reversible. In some cases,

(1N) In some embodiments, the system comprises sensor electrodes and circuits configured to measure one or more electrical signals from any component, compound, or process as described in (1A)-(1M).

### Example 2: Sequencing-by-Synthesis (SBS)

In some embodiments, a method provided herein can comprise using the system as described in (1A)-(1M) of Example 1.

(2A) In some embodiments, the method can comprise applying an electrical field across the membrane, e.g., when at least a portion of the nanopore is engaged on (e.g., embedded in) the membrane. In some cases, the nanopore may not be coupled to any enzyme. In some cases, the nanopore may be coupled to an enzyme, but not coupled to the template-primer complex. In some cases, the nanopore may be coupled to the enzyme-template-primer complex.

(2B) In some embodiments, the method can comprise engaging at least the nanopore to the membrane to effect engaging the nanopore-enzyme-template-primer complex to the membrane, applying the electrical field as provided in (2A), and subsequently, measuring one or more electrical signals from the sensor (e.g., nanopore sensor) while a polymerized product of the enzyme (e.g., growing strand coupled to the primer) is directed into or flowing/translocating through the nanopore.

(2C) In some embodiments, the polymerized product of the enzyme in (2B) can be generated by initiating enzymatic activity of the enzyme (e.g., polymerase activity) on the template (e.g., the target NA molecule) in the presence of the primer.

(2D) In some embodiments, the initiating of the enzymatic activity of the enzyme in (2C) is performed in the presence of the electrical field as provided in (2A) or (2B) (e.g., same electrical field) or a different electrical field.

(2E) In some embodiments, the method comprises forming an enzyme-template-primer complex with a primer overhang (e.g., comprising a polynucleotide sequence that is non-complementary to the template as provided herein), and loading the enzyme-template-primer complex on the nanopore by attracting the primer overhang to the nanopore with the electrical field as provided in (2A) or (2B) (e.g., same electrical field) or a different electrical field. I some cases, the method further comprises initiating the enzymatic activity to generate the growing strand that will be attracted to and translocate through the nanopore, e.g., by the electrical field as provided in (2A) or (2B) or a different electrical field.

(2F) In some embodiments, the method comprises forming an enzyme-template-primer without a primer overhang, and initiating the enzymatic activity to expose the end of the growing strand to be attracted to and translocate through the nanopore.

(2G) In some embodiments, the method comprises forming an enzyme-template-primer complex with a primer overhang, and loading the enzyme-template complex on the nanopore (or the array or nanopores) as the primer overhang is attracted towards the nanopore by the electrical field as provided in (2A) or (2B) or a different electrical field.

(2H) In some embodiments, the method comprises preventing the template (e.g., target NA molecule) to be attracted to the nanopore by adding (e.g., covalently or non-covalently coupling) a heterologous nucleic acid molecule (e.g., a hairpin) that is not the primer NA molecule to one or more ends (e.g., 5′ end or 3′ end) of the template. The heterologous NA molecule can have a length of at least or up to about 2, at least or up to about 5, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, or at least or up to about 100 nucleobases.

(2I) In some embodiments, the method can comprise measuring the electrical signal generated when the polymerized product is attracted to and/or translocating through the nanopore.

(2J) In some embodiments, the method can comprise measuring the electrical signal as described in Example 1 or Example 2, e.g., by measuring current, voltage, impedance, or a change thereof in the sensor. The measuring can be via applying direct current or alternating current.

(2K) In some embodiments, the method can comprise measuring the electrical signal as described in Example 1 or Example 2 multiple times, by driving the polymer through the nanopore back and forth multiple times by alternating the current across the sensor multiple times.

(2L) In some embodiments, the method can comprise using motor enzymes (e.g., the polymerase) as the motor to control the speed of the translocation of the polymerized product through the nanopore.

(2M) In some embodiments, the method can comprise using electrical bias to control the speed of the translocation of polymer through the nanopore (e.g., in the process (2L)).

(2N) In some embodiments, the method can comprise using a nanopore that has a sensor region that has a sensing resolution of a single nucleobase or a combination of two or more nucleobases of the polymerized product.

(2O) In some embodiments, in (2N), the method can comprise using time-resolved electrical signals to differentiate and resolve sensing data indicative of the combination of the two or more nucleobases of the polymerized product, via using direct current or alternating current, thereby achieving a single nucleobase resolution.

(2P) In some embodiments, the method can comprise, in (2N) or (2O), using deconvolution methods to differentiate and resolve the combination of the two or more nucleobases of the polymerized product, via using direct current or alternating current, thereby achieving a single nucleobase resolution. The term “deconvolution”, as used herein, generally refers to a process (e.g., a computerized process) that given the output signal (e.g., sensing data from the combination of the two or more nucleobases) determines two or more unknown input signals (e.g., predicted separate sensing data from each nucleobase of the combination of the two or more nucleobases).

(2Q) In some embodiments of any of the methods in Example 1 or Example 2, the method can comprise reducing the change of or preventing the polymerized product from being fully retracted out of the nanopore (e.g., along a direction opposite of the initial entry direction of the polymerized product to the nanopore), e.g., via coupling a stopper moiety (e.g., a small molecule or a heterologous nucleic acid molecule, etc.) to at least a portion of the polymerized product that reaches the trans chamber (e.g., opposite of the cis chamber comprising the polymerase coupled to the nanopore). In some cases, the stopper moiety can be streptavidin that couples to a biotinylated end of the polymerized product or the primer NA molecule). For example, the non-complementary region of the primer NA molecule can comprise a coupling unit of the stopper moiety (e.g., biotin) prior to forming a complex with the template, such that along sequencing of the complex, the stopper moiety (e.g., streptavidin) can be coupled to the coupling unit when at least a portion of the non-complementary region has translocated to the trans chamber. In some cases, the stopper moiety can be coupled to the 5′ end of the polymerized product or the primer NA molecule. In some cases, the stopper moiety can be coupled to the 3′ end of the polymerized product or the primer NA molecule.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-40. (canceled)

41. A method for analyzing a target nucleic acid molecule, comprising:
(a) providing a complex comprising said target nucleic acid molecule and a primer nucleic acid molecule, wherein said primer nucleic acid molecule comprises (i) a region that is complementary to a portion of said target nucleic acid molecule and (ii) an additional region that is non-complementary to said target nucleic acid molecule;
(b) using said region of said primer nucleic acid molecule and a plurality of nucleotide bases to generate a growing strand, wherein said growing strand is complementary to an additional portion of said target nucleic acid molecule; and
(c) during generating of said growing strand in (b), using a sensor to identify at least said additional region directed through a pore of said sensor, thereby analyzing said target nucleic acid molecule.

42. The method of claim 41, further comprising contacting said complex by an enzyme that is operatively coupled to said pore.

43. The method of claim 42, wherein said contacting directs said additional region through said pore.

44. The method of claim 42, wherein said enzyme is not covalently coupled to said pore.

45. The method of claim 41, wherein (c) further comprises alternating an electrical signal in said sensor to direct said additional region to flow through said pore two or more times.

46. The method of claim 41, wherein, in (a), said region of said primer nucleic acid molecule is flanked by (1) said additional region of said primer nucleic acid molecule and (2) said growing strand.

47. The method of claim 41, wherein (c) comprises identifying a sequence read that comprises a distinctive sequence information that is indicative of at least a portion of said additional region of said primer nucleic acid molecule.

48. The method of claim 41, wherein (c) further comprises obtaining sequence information of at least a portion of said growing strand by directing said at least said portion of said growing strand through said pore of said sensor.

49. The method of claim 48, wherein, in (c), said additional region of said primer nucleic acid molecule is directed through said pore prior to said at least said portion of said growing strand.

50. The method of claim 48, wherein (c) further comprises alternating an electrical signal in said sensor to direct said at least said portion of said growing strand through said pore two or more times.

51. The method of claim 41, wherein said additional region comprises at least 5 bases.

52. The method of claim 41, wherein said additional region comprises at least 10 bases.

53. The method of claim 41, wherein said additional region comprises a net positive charge.

54. The method of claim 41, wherein said additional region comprises a net negative charge.

55. The method of claim 41, wherein said pore is part of a nanopore protein.

56. The method of claim 41, wherein said pore is part of a solid-state nanopore.

57. The method of claim 41, wherein said target nucleic acid molecule is a circular nucleic acid molecule.

58. The method of claim 41, wherein said target nucleic acid comprises a hairpin.

59. The method of claim 41, wherein (c) comprises detecting one or more signals indicative of an impedance or impedance change in said sensor when at least said additional portion is directed through said pore.

60. The method of claim 41, wherein said primer nucleic acid molecule comprises a barcode.
